(19) United States
(12) Patent Application Publication
(10) Pub. No.: US 2008/0300263 A1
(43) Pub. Date: Dec. 4, 2008

(54) Pyrazolo [1,5-a] pyrimidine compounds and pharmaceutical compositions containing them

(75) Inventors: Finbarr Murphy, Cork (GB); Theodore Richard James, Cork (IE); Ian Hayes, Cork (IE)

Correspondence Address: Edwards Angell Palmer & Dodge LLP, P.O. Box 55874, Boston, MA, 02205, US

(73) Assignee: EiRx Therapeutics plc, Cork (IE)

(21) Appl. No.: 12/012,888

(22) Filed: Feb. 6, 2008

(30) Foreign Application Priority Data
Aug. 5, 2005 (GB) 0516378.7
Aug. 8, 2006 (GB) PCT/GB2006/002968

(51) Int. Cl. A61K 31/519; C07D 487/04; A61P 35/00; C12Q 1/02

(52) U.S. Cl. 514/259.3; 544/281; 435/29

(57) ABSTRACT

The present invention relates to certain pyrazolo[1,5a]pyrimidine compounds, to processes for their preparation, compositions comprising them and methods of using them. The compounds are useful in the treatment of cancer. Novel screening methods are also provided.

### FIELD OF THE INVENTION

The present invention relates to certain pyrazolo[1,5-a]pyrimidine compounds, to processes for their preparation, compositions comprising them and methods of using them. Novel screening methods are also provided.

### BACKGROUND AND PRIOR ART

Apoptosis is a genetically regulated process of cell suicide that allows for the removal of unneeded, senescent or infected cells from the body while preserving the integrity and architecture of surrounding tissue. Whereas mitosis is responsible for the generation of new cells, apoptosis in contrast is responsible for removing the cells. It is this delicate balance of mitosis versus apoptosis that maintains tissue homeostasis.

Apoptotic pathways can be sub-divided into two categories, either the extrinsic apoptotic signals which are initiated on the outside of the cell by ligand engagement of cell surface receptors such as Fas and TNF receptors, and/or the intrinsic pathways activated by signals emanating from cellular damage sensors (e.g. p53) or developmental cues. Although the pathways activated by extrinsic and intrinsic signals can overlap to some extent, receptor ligation (via an extrinsic signal) typically leads to recruitment of adapter proteins that promote caspase oligomerization and auto-processing

For example, following Fas ligand binding to the Fas receptor, the death signal is transmitted through conformational changes in preformed receptor clusters, resulting in the recruitment of the adaptor protein FADD (Fas-associated death-domain protein) through a DD-DD interaction. Once bound to Fas to form the death-inducing signalling complex, FADD then binds to the prodomain of caspase-8, which results in autoactivation of caspase-8 by proteolytic processing leading to cellular destruction {reviewed in Murphy et al Curr. Opin Pharm. 2003, 3:412-419).

In contrast, intrinsic signals usually operate by triggering the release of proteins from the mitochondria to the cytosol. Most notable among these is cytochrome c; binding of cytochrome c to a central apoptotic regulator, Apaf-1, promotes oligomerization of Apaf-1 and, in a reaction requiring the ATPase activity of Apaf-1, oligomerization and activation of caspase 9). Caspase 9 subsequently activates effector caspases such as 3, 6, and 7 and cellular destruction ensues (reviewed in Johnson & Jarvis 2004, Apoptosis; 9(4):423-7).

This targeted cell destruction is critical both in physiological contexts as well as pathological states. Apoptosis is the normal physiological response to many stimuli, including irreparable DNA damage. Various diseases evolve because of hyperactivation (neurodegenerative diseases, immunodeficiency, ischaemia-reperfusion injury) or suppression of programmed cell death (cancer, autoimmune disorders).

In cancer, the balance between mitosis and programmed cell death is disturbed, and defects in apoptotic pathways allow cells with genetic abnormalities to survive. The role of apoptosis in the genesis and progression of cancer has been well documented (e.g. Reed J C, 1999; J. Clin Oncol. 17(9), 2941). Failure in normal apoptosis pathways contribute to carcinogenesis by creating a permissive environment for genetic instability and accumulation of gene mutations, promoting resistance to immune-based destruction, allowing disseverance of cell cycle checkpoints that would normally induce apoptosis, facilitating growth factor/hormone-independent cell survival, supporting anchorage-independent survival during metastasis, reducing dependence on oxygen and nutrients, and conferring resistance to cytotoxic, anticancer drugs and radiation. Indeed, studies of colon specimens harvested at various points along the transformation of colorectal epithelium to carcinomas demonstrated a progressive inhibition of apoptosis (Bedi et al, 1995, Cancer Res.; 55(9):1811-6.)

### Colon Cancer

Colorectal cancer is common in economically developed countries, particularly in Europe, North America and Australia and is the second leading causes of cancer-related deaths in the Western world. Every year, colorectal cancer is responsible for an estimated 400,000 deaths worldwide. Approximately 60,000 people die from colorectal adenocarcinoma among the 150,000 new cases, which are diagnosed in Europe each year. A genetic contribution to colon cancer risk is suggested by two observations, namely a) an increased incidence of colorectal cancer among persons with a family history of colorectal cancer and b) families in which multiple family members are affected with colorectal cancer, in a pattern indicating autosomal dominant inheritence of cancer susceptibility (Burt et al 2004, Gastroenterology. 2004; 127(2):444-51; Vasen et al 1996, Lancet. 17; 348(9025):433-5). About 25% of patients with colorectal cancer have a family history of the disease while the remaining 75% of patients have sporadic disease, with no apparent evidence of inheriting the disease.

### Natural History of Colorectal Cancer

Colorectal tumours present with a broad spectrum of neoplasms, ranging from benign growths to invasive cancer and are predominantly epithelial in origin (i.e adenomas or adenocarcinomas). Pathologists have classified the lesion into three groups: nonneoplastic polyps, neoplastic polyps (adenomatous polyps, adenomas) and cancers.

Over 95% of colorectal cancers are carcinomas, most of which are adenocarcinomas. A personal/familial history of having colon adenomas places one at increased risk of developing colon cancer (Neale and Ritchie, in Herrera L, ed. Familial Adenomatous Polyposis. New York, N.Y.; Alan R. Liss Inc., 1990 p61-66) suggesting that either the adenoma may reflect an innate or acquired tendency of the colon to form tumour or that adenomas might be the primary precursor lesion of the colon cancer. While there is no direct proof that the majority of colorectal cancers arise from adenomas, adenocarcinomas are generally considered to arise from adenomas because a) benign and malignant tissue occur within colorectal tumours (Perzin and Bridge 1981, Cancer. 48(3):799-819) and b) when patients with adenomas were followed for 20 years, the risk of cancer at the site of the adenoma was 25%, a rate much higher than the expected norm (Stryker et al 1987, Gastroenterology. 1987; 93(5):1009-13). In addition, removal of adenomatious polyps is associated with reduced colorectal cancer incidence (Muller and Sonnenberg 1995, Ann Intern Med. 15; 123(12):904-10).

### Genesis of Colon Cancer

The colon is organized into compartments of cells called crypts, where stem cells that reside near the bottom give rise to transit amplifying cells that undergo five to seven additional divisions before they become terminally differentiated into one of four cell types, namely colonocytes, goblet cells, Paneth cells and enteroendocrine cells (Brittan & Wright 2004, Gut.; 53(6):899-910). Three of the four cells types (exception being Paneth cells) continue to migrate to the top of the crypt where they undergo apoptosis and are engulfed by stromal cells or are shed into the lumen. For tissue homeostasis to ensue, the birth rate of the colonic epithelial cells precisely equals the rate of loss from the crypt apex. If birth/loss ratio increases, a neoplasm occurs.

Since the differentiated epithelial cells have a fixed residency under normal conditions, it would appear that key to the genesis of colon cancer is that for cells to be able to accumulate mutations and form a polyp and early adenoma in this tissue, they have to become refractory to apoptosis. It is widely believed adenomas develop from normal stem cells through molecular abnormalities (Bach et al 2000, Carcinogenesis. 21(3):469-76). However, a single random major deleterious molecular alteration is not sufficient to induce carcinogenesis. Indeed, even sustained expression of well known oncogenes, including SV40 T antigen, human K-ras Val12 and a dominant negative mutant of human p53 alone or in combination do not lead to adenomas over a 9-12 month period (Kim et al 1993, J Cell Biol; 123(4):877-93). One molecular alteration that seems pivotal to the genesis of colon cancer is the mutation of the APC protein such that approximately 85% of all colon cancers have this protein mutated. In addition, mutation of this gene seems to be one of the earliest events in the colorectal tumour progression pathway (Kinzler & Vogelstein, 1998 in The Genetic Basis of Human Cancer (McGraw-Hill, Toronto).). As such, the APC protein is viewed as a gatekeeper of colon cancer.

### The APC Protein

The adenomatous polyposis coli (APC) encodes a large multidomain protein (310 kDa) that has many different sites for interaction with other proteins expressed constitutively within the normal colonic epithelium and whose activity is modulated by the Wnt signalling pathway (Polakis 2000, Genes Dev. 2000; 14(15):1837-51). The APC protein has been implicated in the regulation of a number of important cellular functions, abnormalities of which have been associated with colon cancer. The best characterized roles for the APC protein in cellular function can be listed as follows:

### APC in the Canonical WNT Pathway

The best-characterized function of APC is as a scaffold protein in a multi-protein complex, consisting of GSK3b, β-catenin (beta-catenin), axin and several phosphatases and kinases and whose activity is modulated by Wnt signalling. Wnts are secreted glycoproteins that act as ligands to stimulate receptor-mediated signal transduction pathways in both vertebrates and invertebrates. Activation of Wnt pathways can modulate cell proliferation, apoptosis, cell behaviours, and cell fate. Wnt signaling pathways often work in a combinatorial manner with other pathways, including the fibroblast growth factor (FGF) and transforming growth factor beta (TGFb) pathways, the retinoid signaling pathways, the p53 pathway and insulin-like growth factor type 1 receptor signaling pathway. The Wnt/beta-catenin pathway is the best understood Wnt signaling pathway and is highly conserved during evolution.

In the presence of a Wnt signal, GSK3b is inactivated, β-catenin degradation does not proceed and thus more β-catenin is available to the cell to prevent apoptosis (van Noort and Clevers 2002; Dev Biol; 244(1):1-8). One proposed mechanism for Wnt-induced dissociation of the GSK3b/Axin/β-catenin complex involves the mammalian family member of proteins termed Frat. The family members are homologous to GSK-3 binding protein (GBO), a protein identified in Xenopus embryos that act as positive regulators of Wnt signaling by stabilizing β-catenin (Yost et al 1998, Cell 93, 1031-1041). In Wnt signaling Frat is thought to be recruited to the GSK-3/Axin/β-catenin complex and cause a dissociation of Axin (Li et al, 1999, EMBO J. 18, 4233-4240). As Axin is a substrate of GSK-3, the dissociation of Axin from GSK-3 results in the dephosphorylation of Axin which decreases its affinity for β-catenin and allows it to be released from the GSK-3/Axin/β-catenin complex (Willert et al 1999, Genes Dev. 13, 1768-1773). To support the role of Frat in Wnt signaling, members of the Frat family of protein are normally expressed at sites of active Wnt signaling (van Amerongen et al, 2005, Genes Dev. 19(4):425-30).

In the absence of extracellular Wnt signalling, GSK3b phosphorylates APC, β-catenin and axin which increases their interaction. This complex results in an increase of a recoginition site for ubiquitination ligases and leads to the destruction of β-catenin by the proteasome, thus decreasing intracellular levels.

In colon cancer, where there are mutations in the APC protein, the β-catenin levels cannot be regulated in this fashion, since the complex of GSK3b, β-catenin, Axin and APC is not formed. Thus β-catenin is not ubiquitinated and subsequently not degraded by the proteasome resulting in increased β-catenin levels that accumulate in the nucleus of the cell.

Nuclear accumulation of β-catenin occurs without a nuclear localization sequence and occurs in a Ran-unassisted manner by direct binding to the nuclear pore machinery (Fagotto et al. 1998 Curr Biol; 8(4):181-90). Beta-catenin is also rapidly transported from the nucleus, thus its nuclear levels are determined by both import and export (Yokoya et al. 1999; Mol Biol Cell; 10(4):1119-31)). In the nucleus β-catenin forms a complex with HMG box transcription factors of the LEF and TCF classes as well as with CREB Binding Protein (CBP, refs. Takemaru and Moon, 2000; J Cell Biol 2000; 149(2):249-54 Hecht et al, 2000; EMBO J; 19(8):1839-50) leading to activation of expression of target genes.

The association of β-catenin with the LEF/TCF transcription factors promote the expression of a large number of genes, some of which have been identified and have been demonstrated to be important in the development and progression of colorectal carcinoma. Such genes include c-myc, cyclin D1, gastrin, cyclooxygenase (COX) −2, matrix metaloproteinase (MMP)-7, urokinase-type plasminogen activator receptor (αPAR), CD44 proteins and β-glycoproteins. Doubtless there are also many more genes that are regulated (both upregulated and directly or consequentially downregulated) by nuclear accumulation of β-catenin that have a fundamental role in genesis, progression and metastasis of colon cancer.

### APC in Cell Division

APC contributes to mitotic spindle formation and function such that during early mitosis, APC localizes to outer kinetochores, consitent with its accumulation at microtubule ends (Kaplan et al 2001; Nat Cell Biol.; 3(4):429-32). Cells lacking APC are more prone to chromosome segregation defects and investigators have recently shown that a single truncating mutation in APC, similar to mutations found in tumor cells, acts dominantly to interfere with microtubule plus-end attachments and cause a dramatic increase in mitotic abnormalities. Researchers propose that APC functions to modulate microtubule plus-end attachments during mitosis and that a single mutant APC allele predisposes cells to increased mitotic abnormalities, which may contribute to tumor progression. They suggest that this is the cause for the high level of chromosomal instability (CIN) observed in colorectal tumour cells (Green and Kaplan, 2003; J. Cell Biol.; 163(5):949-61). In agreement with this is the observation that in premalignant colonic lesions isolated during colonoscopy, there is a correlation between APC mutations in APC and aneuploidy (Nathke, 2004; Annu Rev Cell Dev Biol. 2004; 20:337-66).

### Wnt Signalling in Colon Cancer

Due to the importance of APC in the functioning of the colon epithelium, its association with the genesis of colorectal cancer is not surprising. Truncating mutations in APC have been found in >85% of colon tumours examined (Miyoshi et al 1992; Hum Mol. Genet.; 1(4):229-33). Genetic predisposition to colon cancer is characterized by a germ-line mutation in the APC gene in familial adenomatous polyposis (FAP) in humans (Nishisho et al 1991; Science.; 253(5020):665-9). Individuals carrying a truncating mutation in the APC gene lose the remaining wild type allele, resulting in loss of normal APC function. The multiple intestinal neoplasia (Min) mouse mimics FAP patients in that these mice carry a mutation in one allele of APC and lose the remaining wild-type allele in the tumours that develop (Levy et al 1994; Cancer Res.; 54(22):5953-8). Among other things, the mutant APC protein is defective in its ability to promote proteolytic degradation of β-catenin and leads to an activation of β-catenin induced transcription.

In support of this, overexpression of β-catenin has been shown to promote cell proliferation and colony growth in soft agar (Orford et al 1999; J. Cell Biol.; 146(4):855-68; Wagenaar et al, 2001; Cancer Res. 2001; 61 (5):2097-104). Conversely, induction of wild type (wt)-APC protein in the colorectal cancer cell line HT-29, using an inducible expression system, resulted in substantial reduction in growth and an induction of apoptosis (Morin et al 1999, Carcinogenesis.; 20(11):2045-9; Hsi et al 1996 Proc Natl Acad Sci USA, 23; 93(15):79504.) In addition, overexpression of wt-APC in another colon cell line SW480 resulted in changes in cell morphology, actin cytosleketon as well as reduced proliferation and reduced ability to form colonies in soft agar and do not grow in a xenograft mouse tumour model—thus indicating reduction in the tumorigenic phenotype (Faux et al 2004; J Cell Sci.; 117(Pt 3):427-39). Likewise, antisense oligonucleotides that down regulate levels of β-catenin inhibit cell proliferation and anchorage independent growth of SW480 cells (Roh et al 2001; Cancer Res. 2001 Sep. 1; 61(17):6563-8).

However the consensus is that while mutations in APC or β-catenin are sufficient to initiate the growth of a small benign tumour they are not sufficient to make such tumours progress to more advanced forms. Several other pathways participate in this progression. One of these pathways involves transforming growth factor β (TGFβ), a small polypeptide hormone that negatively controls colon cell growth through regulation of transcription factors like SMAD4. A second critical pathway involved in tumor progression involves p53, a gene that is inactivated not only in colorectal cancers but also in most other cancer types. Activation of the normal p53 gene inhibits cell growth by blocking the cell cycle and by stimulating cellular suicide (apoptosis).

The final stage of the tumourigenic process, metastasis, is responsible for most deaths from cancer. Recently Vogelstein et al (Clin Cancer Res. 2003 Nov. 15; 9(15):5607-15; Cancer Cell. 2005 June; 7(6):561-73) discovered two genes that appear to be important for this lethal event. One is PRL-3, which encodes a protein tyrosine phosphatase that is highly expressed in metastatic colorectal cancer cells but not in earlier stages of the disease. The second is PIK3CA, which encodes a lipid kinase that is mutated in invasive forms of colorectal cancers as well as several other invasive cancer types (including cancers of the breast, stomach, brain, ovary, and lung). The delineation of the molecular events of colon cancer offers new therapies in the future.

### Wnt Signaling in Breast Cancer

Anomalous Wnt signaling has been implicated in a number of studies as a causative factor in the development of breast cancer. Early work by Nusse and Varmus demonstrated that the mouse mammary tumour virus (MMTV) acts as an insertional mutagen in mouse mammary tissue and that in many MMTV-induced tumours, integration of the proviral DNA resulted in transcriptional activation of the Wnt1 gene ((Nusse & Varmus; Cell 1-992, 69:1073-1087). Further evidence that activation of the canonical Wnt signaling pathway promotes tumorigenesis in mouse mammary tissues include the fact that certain strains of mice carrying a germline truncation mutation in APC protein show enhanced sensitivity to carcinogen-induced mammary tumors (Moser et al., Cancer Res 2001, 61: 3480-3485) while transgenic mice expressing stabilized β-catenin in the mammary gland develop carcinomas (Imbert et al, J Cell Biol 2001, 153:555-568).

In humans, Cyclin D1 overexpression has been found in approximately 50% of patients with breast cancer (Gillet et al 1994, Cancer Research, 54, 7, 1812-1817). By analyzing the promoter region of cyclin D1, a perfect T cell factor 4 (Tcf4)-binding site (CTTTGATC) was identified suggesting the potential involvement of the β-catenin/Tcf4 pathway in the regulation of cyclin D1 expression (Lin et al 2000, 97, 8, 4262-4266). In agreement with this, is the finding that elevated β-catenin-Tcf transcriptional activity has been observed in certain breast cancer cell lines, with the MCF7 breast cell line having the highest activity of eight breast cancer cell lines examined. In addition, nuclear and cytoplasmic staining of β-catenin has been reported in as many as 60% of human breast cancer specimens. Most importantly, this staining pattern is an independent marker that correlates with poor prognosis (Lin et al 2000, PNAS, 97, 8 4262-4266). More recently, the CC-chemokine Monocyte chemotactic protein-1 (MCP-1/CCL2), which has been implicated in tumour progression events such as angiogenesis and tumour associated macrophage infiltration, was shown to be regulated in breast cancer cells by the β-catenin pathway (Mestdagt et al, 2005, Int J. Cancer, July, in press).

Additionally, overexpression of an inactive form of glycogen synthase kinase 3β (GSK3β), which stabilizes the level of β-catenin, resulted in the development mammary tumours, when expressed in the mammary gland of the mouse (Farago et al, Cancer Res. 2005, 65(13) 5792-801). In agreement with this, in an in vitro transformation model, an increase in β-catenin expression was observed in aggressive tumourigenic breast cells relative to parental non transformed MCF10F cells, thus demonstrating a role for β-catenin in breast cell transformation (Calaf et al. Int J Oncol 2005, 26(4) 913-21).

In conclusion therefore, the role of aberrant Wnt signaling and anomalous β-catenin expression is well documented for Breast cancer.

### Wnt-Signaling in Other Cancers

Oncogenic mutations of the APC protein or β-catenin have been found in a variety of human cancers, all of which result in a constitutively stable β-catenin protein. As well as colorectal, these cancers also include medulloblastomas, hepatoblastomas, hepatocellular carcinomas, pilomaticomas, endometrial, thyroid and Wilms' tumours (Polakis P. Genes Dev 2000 14, 1837-1851). These data imply that activation of the Wnt signalling pathway, by one means or another is one of the most common signalling abnormalities known in human cancer (Brown A, 2001, Breast Cancer Res 3: 351-355).

### FRAT2 (Frequently Rearranged in Advanced T-Cell Lymphomas 2)

Saitoh et al cloned FRAT2 cDNAs, spanning the complete coding sequence, from a human fetal lung cDNA library. FRAT2 encoded 233 amino-acid protein, which showed 77.3% total amino-acid identity with Frat1; Frat2 and Frat3 were more homologous in the acidic domain (96% identity), the proline-rich domain (92% identity), and the GSK-3beta binding domain (100% identity). Elevated expression levels of Frat2 is associated with several forms of cancer, in particular gastric cancer (Saitoh et al 2001; Biochem Biophys Res Commun; 281(3):815-20).

Both Frat1 and Frat2 are intronless genes localized to the same portion of chromosome 10q24.1 and separated by only 10.7 kb (Freemantle et al, 2002; Gen; 291(1-2):17-27). In a broad range of human tissues Frat1 and Frat2 are readily detected and expressed in a near identical pattern. The overlapping expression patterns suggest these two genes share a regulatory region and have similar function.

The proto-oncogene Frat1 was originally identified as a common site of proviral insertion in transplanted tumors of Moloney murine leukemia virus (M-MuLV)-infected Emu-Pim1 transgenic mice. Contrary to most common insertion sites implicated in mouse T cell lymphomagenesis, retroviral insertional mutagenesis of Frat1 constitutes a relatively late event in M-MuLV-induced tumor development, suggesting that proviral activation of Frat1 contributes to progression of T cell lymphomas rather than their genesis. To substantiate this notion researchers generated transgenic mice that overexpress Frat1 in various organs, including lymphoid tissues. Frat1 transgenic mice develop focal glomerulosclerosis and a nephrotic syndrome, but they do not exhibit an increased incidence of spontaneous lymphomas. Conversely, these mice are highly susceptible to M-MuLV-induced lymphomagenesis, and Frat1/Pim1 bitransgenic animals develop lymphomas with increased frequency compared to Pim1 transgenic littermates. These data support a role for Frat1 in tumor progression (Jonkers et al, 1999; Oncogene; 18(44):5982-90). Frat1-deficient mice in which most of the coding domain was replaced by a promoterless beta-galactosidase reporter gene are normal, healthy and fertile. The pattern of LacZ expression in Frat1(lacZ)/+ mice indicated Frat1 to be expressed in various neural and epithelial tissues.

Over-expression of Frat1 and Frat2 leads to β-catenin stabilization through disassociation of GSK3β from Axin and inhibition of β-catenin phosphorylation. Consequently, Frat1 and Frat2 are positive regulators of the WNT-β-catenin signaling pathway. Culbert et al compared neuroprotection resulting from modulation of GSK-3 activity in PC12 cells using either selective small molecule ATP-competitive GSK-3 inhibitors (SB-216763 and SB-415286), or adenovirus overexpressing FRAT1, Cellular overexpression of FRAT1 is sufficient to confer neuroprotection and correlates with inhibition of GSK-3 activity towards beta-catenin, but not modulation of glycogen synthase (GS) activity. By comparison, treatment with SB-216763 and SB-415286 proved more potent in terms of neuroprotection, and correlated with inhibition of GSK-3 activity towards GS in addition to beta-catenin. (FEBS Lett 2001 2; 507(3):288-94).

### Current Treatment of Colorectal Cancer

Current best practice for the treatment of colon cancer is resection of the affected area. However, the success of this treatment regime is solely based on the early presentation and diagnosis of the patient. Thus early diagnosis results in the best prognosis.

5-Fluorouracil (5-FU) and irinotecan have to date been the most widely used single agent therapies in the treatment of advanced metastatic colon cancers. Upon cell entry, 5-FU is converted to its active form 5-fluoro-20-deoxyuridine monophosphate (Allegra C J, Grem J L (1997) Antimetabolites. In Cancer: Principles and Practice of Oncology, DeVita V T, Hellman S, Rosenberg S A (eds) 5th edn, pp 432-452. Philadelphia: Lippincott-Raven), whereby a complex is formed with thymidylate synthase (Santi et al, 1987), inhibiting its function and impairing DNA synthesis. 5-Fluorouracil is also incorporated into RNA and interferes with RNA processing. It is an S-phase active agent with no activity in G0 or G1 and causes S-phase arrest (Santi et al, 1987, Biochemistry 26: 8606-8613). Irinotecan (CPT-11) is a water-soluble camptothecin analogue, which inhibits topoisomerase I via conversion to its active metabolite SN38. SN38 inhibits topoisomerase I activity by stabilising the topoisomerase I-DNA cleavable complex, which results in DNA double-strand breaks and ultimately to cell death. Cells in S-phase are significantly more sensitive to camptothecins than cells in G1 or G2 (Li et al, 1972; Cancer Res 32: 2643-2650).

More recently the FDA has approved monoclonal antibodies targeting specific epitopes on cancer cells for use against colon cancers. Two such therapies include Avastin (Genentech) and Erbitux (ImClone).

Avastin works against colon cancer by blocking a protein called vascular endothelial growth factor (VEGF). Tumors need the protein to grow and maintain their blood vessels. When VEGF is blocked, the tumor gets less blood, so it shrinks or spreads more slowly. Patients receiving Avastin lived about five months longer than patients on the standard regimen alone.

Erbitux is a genetically engineered version of a mouse antibody that contains both human and mouse components. This new monoclonal antibody is believed to work by targeting epidermal growth factor receptor (EGFR) on the surface of cancer cells, interfering with their growth.

For patients with tumors that express EGFR and who no longer responded to treatment with irinotecan alone or in combination with other chemotherapy drugs, the combination treatment of Erbitux and irinotecan shrank tumors in 22.9% of patients and delayed tumor growth by approximately 4.1 months. For patients who received Erbitux alone, the tumor response rate was 10.8% and tumor growth was delayed by 1.5 months.

The current chemoprophylaxis regimes have arisen more through anecdotal evidence than hard science. Three of the best-characterised treatments are listed below. However, while none represent ideal intervention, clinical trials have demonstrated proof of principal that it is possible to intervene and reduce the risk of colon cancer.

### a) Aspirin

Use of aspirin and other NSAIDs is associated with reduced risk of colorectal carcinomas and cancer. Despite this benefit, aspirin is associated with significant risk of Gastrointestinal (G1) hemorrhage, as well as renal and hepatic side effects. Recently use of aspirin among women was associated with increased risk of pancreatic cancer.

### b) COX-2 Specific Inhibitors

To overcome the nonspecific effect of aspirin, several pharmaceutics companies have developed COX-2 specific inhibitors. In 1999, the FDA granted accelerated marketing approval for celecoxib “to reduce the number of adenomatous colorectalpolyps in FAP as an adjunct to usual care” (FDA, Dec. 23, 1999). The rationale for targeting COX-2 specifically was the observation that COX isozymes were over expressed in colorectal adenomas and cancers as well as the fact that that targeted deletion of COX-2 gene prevented colorectal cancer in animals.

However, despite the promise of efficacy and the widespread safe use of COX-2 specific inhibitors, long term safety of these drugs has not yet been established. COX-2 inhibitors appear to share the same side effects as that of aspirin, although the G1 effects appear to be less severe than aspirin. However, one of the most worrying side effects is the heightened risk of myocardial infarction (Ml). Currently, a number of manufacturers are reviewing their safety data on these types of drugs, with at least one major pharmaceutical company having to take their product off the market.

### c) Ursodiol

Several lines of evidence indicate that fecal bile acids are important promoters of colon cancer. Ursodeoxycholic acid (ursodiol) has been shown to inhibit experimental colon carcinogenesis at least partly through decreasing toxic secondary bile acids. The efficacy of ursodiol against colonic neoplasia in patients with ulcerative colitis and primary sclerosing cholangitis has been demonstrated. Other activities of ursodiol that may contribute to it chemopreventative efficacy include inhibition of colon epithelium proliferation and induction of apoptosis. Ursodiol is currently in a phase III study to evaluates its efficacy in adenoma prevention.

Unfortunately, the limitations of the current treatment and prophylaxis regimes are highlighted by the fact that every year, colorectal cancer is responsible for an estimated 400,000 deaths worldwide. Approximately 60,000 people die from colorectal adenocarcinoma among the 150,000 new cases, which are diagnosed in Europe each year.

There is an urgent need to develop therapeutics to induce apoptosis in the tumour. There are no chemotherapy agents that are specific for colon adenomocarcinomas, thus once the tumour has developed to a certain stage, prognosis is very poor.

### Kinase Based Therapies

Protein kinases are critical components of cellular signal transduction cascades. They are directly involved in apoptosis and survival pathways and as such are implicated in many diseases such as cancer and inflammation. Consequently they have become one of the most important target classes for drug development (Cohen P Nat. Rev. Drug Discov. 1, 309-315 (2002)). The approval of imatinib (Gleevec) for chronic myeloid leukemia (CML) and gefitinib (Iressa) and erlotinib (Tarceva) for non-small cell lung cancer (NSCLC) has provided proof-of-principle that small molecule kinase inhibitors can be effective drugs. Currently, approximately 37 kinase inhibitors are currently in clinical development, with many more being in preclinical studies.

### PRIOR ART

Pyrazolopyrimidines are known. For example, WO92/18504, WO02/50079, WO95/35298, WO02/40485, EP94304104.6, EP0628559, U.S. Pat. No. 6,383,790, Chem. Pharm. Bull., (1999) 47 928, J. Med. Chem., (1977) 20,296, J. Med. Chem., (1976)19 517 and Chem. Pharm. Bull., (1962) 10 620 disclose various pyrazolopyrimidines.

WO2004/022561 discloses pyrazolo[1,5-a]pyrimidine compounds stated to be cyclin-dependent kinase inhibitors. An illustrative example has the formula

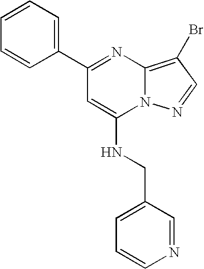

WO20041022560 discloses pyrazolo[1,5-a]pyrimidine compounds stated to be cyclin-dependent kinase inhibitors.

WO2004/026229 discloses pyrazolo[1,5-a]pyrimidine compounds stated to be cyclin-dependent kinase inhibitors.

WO2004/076458 discloses pyrazolo[1,5-a]pyrimidine compounds stated to be kinase inhibitors.

US200410209878 discloses pyrazolo[1,5-a]pyrimidine compounds stated to be cyclin-dependent kinase inhibitors.

WO2005/063756 discloses pyrazolo[1,5a]pyrimidine compounds stated to be corticotrophin-releasing factor antagonists.

J. Med. Chem. 48, 2005, 7604-7614 discloses pyrazolo[1,5-a]pyrimidine compounds as inhibitors of human protooncogene kinase PIM-1.

WO20041087707 discloses pyrazolo[1,5a]pyrimidine compounds stated to be kinase inhibitors.

The present invention addresses I alleviates the problems of the prior art.

### SUMMARY OF THE INVENTION

According to a first aspect, the invention provides a compound of the formula (I):

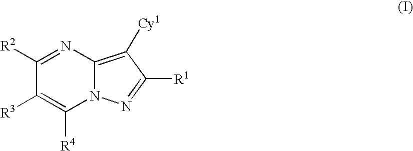

wherein
Cy¹ is an optionally substituted mono or bicyclic aromatic group of from 5 to 10 ring members and 1 to 10 carbon atoms, optionally having from 1 to 5 heteroatoms independently selected from sulphur, oxygen and nitrogen;
R¹, R² and R³ are each independently selected from hydrogen, hydroxyl, halogen, alkyl of 1 to 6 carbon atoms, haloalkyl of 1 to 6 carbon atoms comprising from 1 to a maximum number of halogen atoms, cycloalkyl of 3 to 8 carbon atoms, alkoxy of 1 to 6 carbon atoms, cycloalkoxy of 3-8 carbon atoms, haloalkoxy of 1 to 6 carbon atoms comprising from 1 to a maximum number of halogen atoms, thioalkyl of 1 to 6 carbon atoms, sulfoxoalkyl of 1 to 6 carbon atoms, sulfonoalkyl of 1 to 6 carbon atoms, aryl of 6 to 10 carbon atoms, —COR⁵, CO₂R⁵, —NO₂, —CONR⁵R⁶, —NR⁵R⁶ or —N(R⁵)COR⁶, —CN, a 5 or 6 membered heterocyclic ring having from 1 to 4 heteroatoms selected from O, N or S; —NO₂, —NR⁵R⁶, —CHFCN, —CF₂CN, alkynyl of 2 to 7 carbon atoms, or alkenyl of 2 to 7 carbon atoms; wherein the alkyl, heterocyclic ring, alkenyl or alkynyl moieties are optionally substituted with hydroxyl, —CN, halogen, alkyl of 1 to 6 carbon atoms, alkoxy of 1 to 6 carbon atoms, —COR⁵, —CO₂R⁵, —NO₂, CONR⁵R⁶—NR⁵R⁶ or —N(R⁵)COR⁶;
R⁴ is a group of the formula —NH—CH₂—Cy²;
R⁵ and R⁶ are each, independently hydrogen, alkyl of 1 to 6 carbon atoms or aryl of 6-10 carbon atoms;
Cy² is an optionally substituted cyclic group;
or a pharmaceutically acceptable salt form thereof;
for use as a medicament.

According to a second aspect of the invention, there is provided a compound of the formula (II)

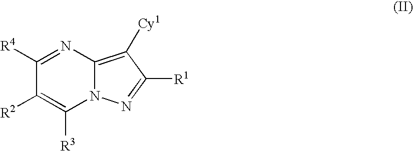

wherein
Cy¹ is an optionally substituted mono or bicyclic aromatic group of from 5 to 10 ring members and 1 to 10 carbon atoms, optionally having from 1 to 5 heteroatoms independently selected from sulphur, oxygen and nitrogen;
R¹, R² and R³ are each independently selected from hydrogen, hydroxyl, halogen, alkyl of 1 to 6 carbon atoms, haloalkyl of 1 to 6 carbon atoms comprising from 1 to a maximum number of halogen atoms, cycloalkyl of 3 to 8 carbon atoms, alkoxy of 1 to 6 carbon atoms, cycloalkoxy of 3-8 carbon atoms, haloalkoxy of 1 to 6 carbon atoms comprising from 1 to a maximum number of halogen atoms, thioalkyl of 1 to 6 carbon atoms, sulfoxoalkyl of 1 to 6 carbon atoms, sulfonoalkyl of 1 to 6 carbon atoms, aryl of 6 to 10 carbon atoms, —COR⁵, —CO₂R⁵, —NO₂, —CONR⁵R⁶, —NR⁵R⁶ or —N(R⁵)COR⁶, —CN, a 5 or 6-membered heterocyclic ring having from 1 to 4 heteroatoms selected from O, N or S; —NO₂, —NR⁵R⁶, —CHFCN, —CF₂CN, alkynyl of 2 to 7 carbon atoms, or alkenyl of 2 to 7 carbon atoms; wherein the alkyl, heterocyclic ring, alkenyl or alkynyl moieties are optionally substituted with hydroxyl, —CN, halogen, alkyl of 1 to 6 carbon atoms, alkoxy of 1 to 6 carbon atoms, —COR⁵, —CO₂R⁵, —NO₂, —CONR⁵R⁶, —NR⁵R⁶ or —N(R⁵)COR⁶;
R⁴ is a group of the formula —NH—Cy²;
R⁵ and R⁶ are each, independently hydrogen, alkyl of 1 to 6 carbon atoms or aryl of 6-10 carbon atoms;
Cy² is an optionally substituted cyclic group;
or a pharmaceutically acceptable salt form thereof;
for use as a medicament.

According to a third aspect, the invention provides a pharmaceutical composition comprising a compound of formula (I) or (II) together with at least one pharmaceutically acceptable carrier.

According to a fourth aspect, the invention provides a method of treating, inhibiting or preventing cancer in a mammal in need thereof, which comprises providing to said mammal an effective amount of a compound of formula (I) or (II).

According to a fifth aspect, the invention provides a method of inducing, inhibiting or modulating apoptosis in a mammal comprising providing to said mammal an effective amount of a compound of formula (I) or (II).

According to a sixth aspect, the invention provides the use of compound of formula (I) or (II) in a process for the preparation of a medicament for treating, inhibiting or preventing cancer.

According to a seventh aspect, the invention provides the use of compound of formula (I) or (II) in a process for the preparation of a medicament for treating, inhibiting or preventing a disease which is alleviated by the induction of apoptosis.

According to an eighth aspect, there is provided a method for detecting apoptosis-modulating activity in a candidate compound comprising the steps of:
i) providing a reference cell line;
ii) providing the reference cell line transformed to overexpress the FRAT2 gene (transformed cell line);
iii) incubating a candidate compound with a) the reference cell line, b) the transformed cell line in the absence of GM-CSF, and c) the transformed cell line in the presence of GM-CSF;
iv) quantifying the proportion of cells killed in cases a), b) and c);
v) comparing the proportion of cells killed in cases a), b) and c); wherein the proportion killed in c) being greater than the proportion killed in a) and the proportion killed in b) being greater than the proportion killed in c) being indicative of apoptosis-modulating activity.

### BRIEF DESCRIPTION OF THE FIGURES

FIGS. 1 to 20 are graphs

### DETAILED DESCRIPTION OF THE INVENTION

### Group Cy¹

Preferably, Cy¹ comprises from 1 to 5 heteroatoms (that is, at least one of the rings of Cy¹ is heterocyclic) independently selected from the group consisting of nitrogen, oxygen, and sulphur. Preferably, Cy¹ comprises 1 heteroatom. Preferably, the heteroatom is nitrogen.

In an alternative preferred embodiment, Cy¹ does not comprise heteroatoms (that is the ring system of Cy¹ is carbocyclic). The substituents of Cy¹ may however comprise such heteroatoms.

At least one of the rings of Cy¹ is an aromatic ring. Aromatic, as used herein, refers to a ring structure that has (4n+2) π electrons, where n is an integer. If Cy¹ comprises more than one ring, some, none or all of said rings may be aromatic. More preferably, all the rings Cy¹ are aromatic.

Preferably, Cy¹ comprises at least one carbocyclic ring (i.e. a ring comprising only carbon atoms). An example of a carbocyclic ring is a cycloalkyl group. Preferred cycloalkyl groups are those having from 3 to 12, more preferably from 3 to 7 carbon atoms.

The group Cy¹ may feature any degree of unsaturation consistent with a stable chemical structure. For example, there may be one or more than one double bond, one or more than one triple bond, or both kinds of bond.

Where Cy¹ comprises more than one ring, the rings may be fused in any chemically stable configuration, especially ortho-fused (two rings share two atoms), or spiro fused (two rings share one atom). Bridged structures (such as quinuclidine) are also envisaged.

Examples of the aforementioned aromatic groups are thiophene, furan, isobenzofuran, chromene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalizine, naphthyridine, quinoxaline, quinazoline, cinnoline, pteridine, phenyl and naphthyl.

It is very highly preferred that Cy¹ is an optionally substituted phenyl, thiophene (especially 2-thiophene), pyridine (especially 4-pyridine) or quinoline (especially 8-quinoline) group.

Preferred optional substituents of Cy¹ are from 1 to 5 substituents independently selected from the group consisting of hydroxyl, halogen, alkyl of 1 to 6 carbon atoms, haloalkyl of 1 to 6 carbon atoms comprising from 1 to a maximum number of halogen atoms, cycloalkyl of 3 to 8 carbon atoms, alkoxy of 1 to 6 carbon atoms, cycloalkoxy of 3-8 carbon atoms, haloalkoxy of 1 to 6 carbon atoms comprising from 1 to a maximum number of halogen atoms, thioalkyl of 1 to 6 carbon atoms, sulfoxoalkyl of 1 to 6 carbon atoms, sulfonoalkyl of 1 to 6 carbon atoms, aryl of 6 to 10 carbon atoms, —COR¹, —CO₂R⁸, —NO₂, —CONR⁸R⁹, —NR⁸R⁹ or —N(R⁸)COR⁹, —CN, a 5 or 6-membered heterocyclic ring having from 1 to 4 heteroatoms selected from O, N or S; —NO₂, —NR⁸R⁹, —CHFCN, —CF₂CN, alkynyl of 2 to 7 carbon atoms, or alkenyl of 2 to 7 carbon atoms; wherein the alkyl, heterocyclic ring, alkenyl or alkynyl moieties are optionally substituted with hydroxyl, —CN, halogen, alkyl of 1 to 6 carbon atoms, alkoxy of 1 to 6 carbon atoms, —COR⁸, —CO₂R⁸, —NO₂, —CONR⁸R⁹, —NR⁸R⁹ or —N(R⁸)COR⁹;

R⁸ and R⁹ are each, independently hydrogen, alkyl of 1 to 6 carbon atoms or aryl of 6-10 carbon atoms.

Highly preferred optional substituents of Cy¹ are from 1 to 5 groups independently selected from alkoxy of 1 to 6 carbon atoms (especially methoxy), haloalkoxy of 1 to 6 carbon atoms comprising from 1 to a maximum number of halogen atoms (especially chloromethyl), halogen (especially fluoro and chloro), —NR⁸R⁹, and a 5 or 6-membered heterocyclic ring having from 1 to 4 heteroatoms selected from O, N or S (especially morpholine, more especially 4-morpholine).

Very preferably, Cy¹ is selected from 3-methoxyphenyl, 3,4-dimethoxyphenyl, 4-methoxyphenyl, 2,4-dimethoxyphenyl, phenyl, 2,4-fluorophenyl, 3-(dimethylamino)phenyl, 3-quinolin-8-yl, 6-methoxy-pyridin-3-yl, 4-methyl-thiophen-2-yl, 3-chloro-4-fluorophenyl, 2-(chloromethyl)phenyl, morpholin-4-yl-phenyl and 4-pyridyl.

### Groups R¹, R² and R³

Preferably, at least one of R¹, R² and R³ is hydrogen. Preferably, R¹ is hydrogen. Preferably, R² is hydrogen. Preferably, R³ is hydrogen. Preferably at least two of R¹, R² and R³ are hydrogen. More preferably, all of R¹, R² and R³ are hydrogen.

### Group R⁴

### Group Cy²

Preferred compounds of the invention are compounds of formula (I) wherein Cy² is an optionally substituted mono, bi- or tricyclic group.

Preferably, Cy² comprises from 1 to 6 heteroatoms (that is, at least one of the rings of Cy² is heterocyclic) independently selected from the group consisting of nitrogen, oxygen, sulphur and phosphorous.

In an alternative preferred embodiment, Cy² does not comprise heteroatoms (that is the ring system of Cy² is carbocyclic). The substituents of Cy² may however comprise such heteroatoms.

Preferably, at least one of the rings of Cy² is an aromatic ring. Aromatic, as used herein, refers to a ring structure that has (4n+2) π electrons, where n is an integer. If Cy² comprises more than one ring, some, none or all of said rings may be aromatic. More preferably, all the rings Cy² are aromatic.

Preferably, Cy² comprises at least one carbocyclic ring (i.e. a ring comprising only carbon atoms). An example of a carbocyclic ring is a cycloalkyl group. Preferred cycloalkyl groups are those having from 3 to 12, more preferably from 3 to 7 carbon atoms.

The group Cy² may feature any degree of unsaturation consistent with a stable chemical structure. For example, there may be one or more than one double bond, one or more than one triple bond, or both kinds of bond.

Where Cy² comprises more than one ring, the rings may be fused in any chemically stable configuration, especially ortho-fused (two rings share two atoms), or spiro fused (two rings share one atom). Bridged structures (such as quinuclidine) are also envisaged.

In a preferred embodiment, Cy² is an optionally substituted mono or bicyclic aromatic group of from 1 to 10 carbon atoms, optionally having from 1 to 5 heteroatoms selected from sulphur, oxygen and nitrogen.

Examples of the aforementioned aromatic groups are thiophene, furan, isobenzofuran, chromene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalizine, naphthyridine, quinoxaline, quinazoline, cinnoline, pteridine, phenyl and naphthyl.

It is very highly preferred that Cy² is an optionally substituted thiophene (especially 2-thiophene) or pyridine (especially 2-pyridine) group.

Optional-substituents of Cy² are from 1 to 5 substituents independently selected from the group consisting of hydroxyl, halogen, alkyl of 1 to 6 carbon atoms, haloalkyl of 1 to 6 carbon atoms comprising from 1 to a maximum number of halogen atoms, cycloalkyl of 3 to 8 carbon atoms, alkoxy of 1 to 6 carbon atoms, cycloalkoxy of 3-8 carbon atoms, haloalkoxy of 1 to 6 carbon atoms comprising from 1 to a maximum number of halogen atoms, thioalkyl of 1 to 6 carbon atoms, sulfoxoalkyl of 1 to 6 carbon atoms, sulfonoalkyl of 1 to 6 carbon atoms, aryl of 6 to 10 carbon atoms, —COR⁸, —CO₂R⁸, —NO₂, —CONR⁸R⁹, —NR⁸R⁹ or —N(R⁸)COR⁹, —CN, a 5 or 6-membered heterocyclic ring having from 1 to 4 heteroatoms selected from O, N or S; —NO₂, —NR⁸R⁹, —CHFCN, CF₂CN, alkynyl of 2 to 7 carbon atoms, or alkenyl of 2 to 7 carbon atoms; wherein the alkyl, heterocyclic ring, alkenyl or alkynyl moieties are optionally substituted with hydroxyl, —CN, halogen, alkyl of 1 to 6 carbon atoms, alkoxy of 1 to 6 carbon atoms, —COR⁸, —CO₂R⁸, —NO₂, —CONR⁸R⁹, —NR⁸R⁹ or —N(R⁸)COR⁹;

R⁸ and R⁹ are each, independently hydrogen, alkyl of 1 to 6 carbon atoms or aryl of 6-10 carbon atoms.

Preferably, Cy² does not have substituents.

Very highly preferred compounds of the invention have the formula (III)

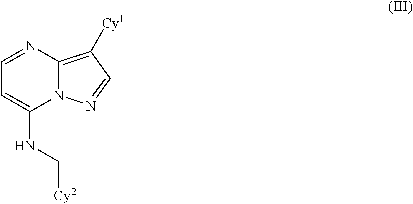

wherein Cy¹ and Cy² are as defined above.

Alternative very highly preferred compounds of the invention have the formula (IV)

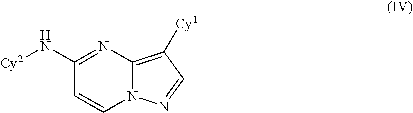

wherein Cy¹ and Cy² are as defined above.

### Preferred Compounds

Among the specifically preferred compounds of the invention are:

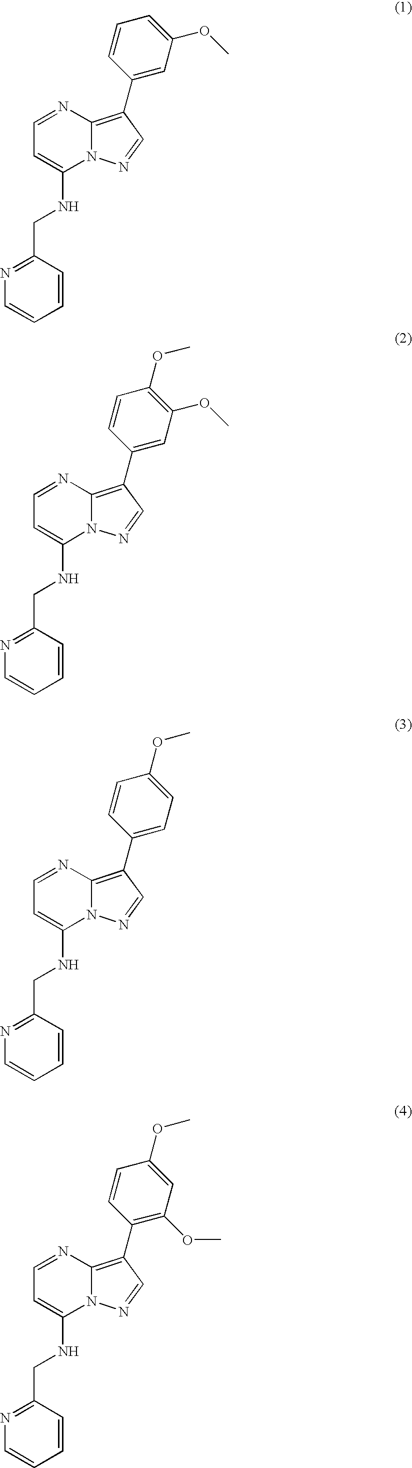

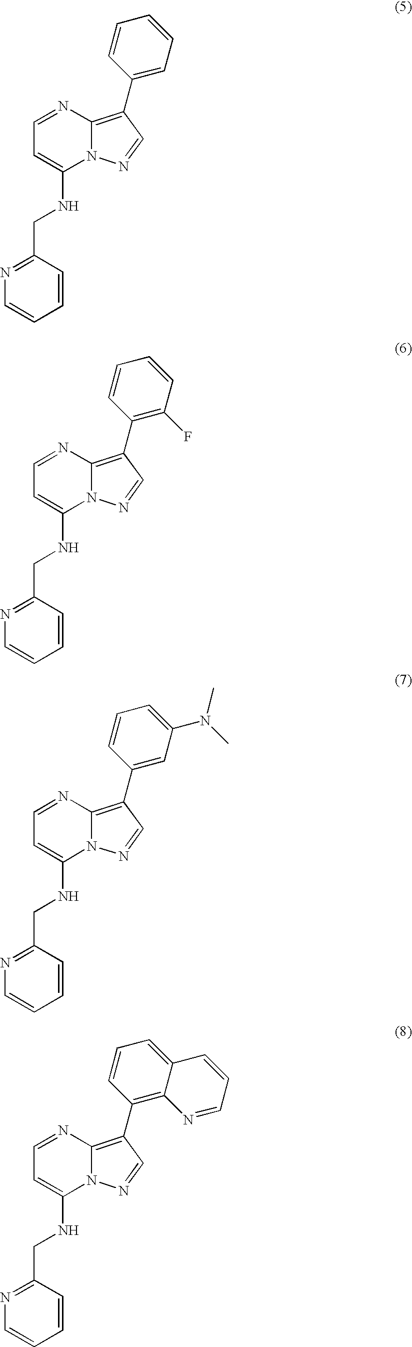

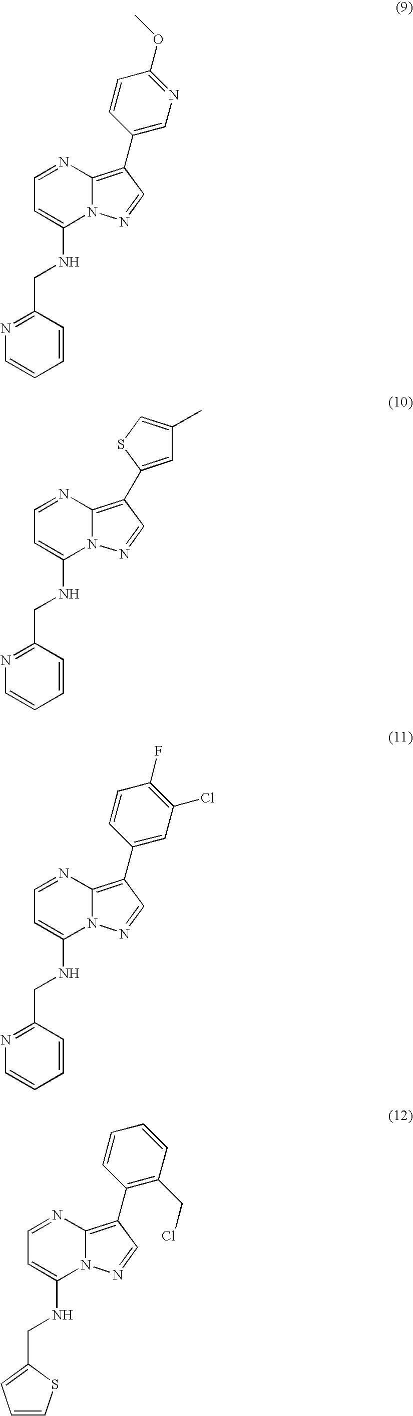

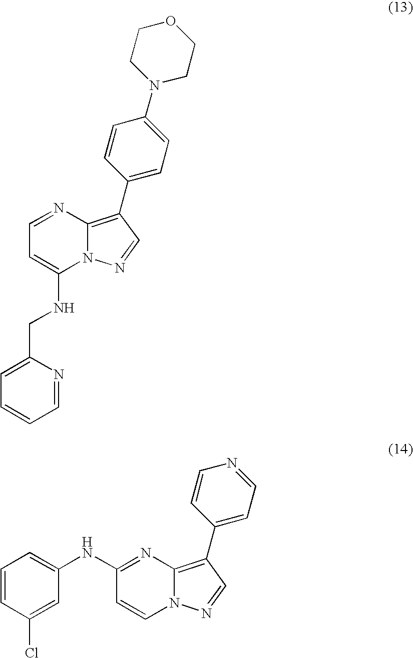

### DEFINITIONS

### Alkyl

Alkyl, as used herein refers to an aliphatic hydrocarbon chain and includes straight and branched chains e.g. of 1 to 6 carbon atoms such as methyl, ethyl, n-propyl, isopropyl, n-butyl, isobutyl, sec-butyl, t-butyl, n-pentyl, isopentyl, neo-pentyl, n-hexyl, and isohexyl.

### Alkenyl

Alkenyl, as used herein, refers to an aliphatic hydrocarbon chain having at least one double bond, and preferably one double bond, and includes straight and branched chains e.g. of 2 to 6 carbon atoms such as ethenyl, propenyl, isopropenyl,but-1-enyl, but-2-enyl, but-3-enyl, 2-methypropenyl.

### Alkynyl

Alkynyl, as used herein, refers to an aliphatic hydrocarbon chain having at least one triple bond, and preferably one triple bond, and includes straight and branched chains e.g. of 2 to 6 carbon atoms such as ethynyl, propynyl, but-1-ynyl, but-2-ynyl and but-3-ynyl.

### Cycloalkyl

Cycloalkyl, as used herein, refers to a cyclic, saturated hydrocarbon group having from 3 to 8 ring carbon atoms. Examples of cycloalkyl groups are cyclopropyl, cyclobutyl, cyclopentyl, cyclohexyl, cycloheptyl and cyclooctyl.

### Alkoxy

Alkoxy as used herein refers to the group alkyl, wherein alkyl is as defined above. Examples of alkoxy groups include methoxy, ethoxy, n-propoxy, isopropoxy, n-butoxy, isobutoxy, sec-butoxy, t-butoxy, n-pentoxy, isopentoxy, neo-pentoxy, n-hexyloxy, and isohexyloxy.

### Cycloalkoxy

Cycloalkoxy as used herein refers to the group β-cycloalkyl, wherein cycloalkyl is as defined above. Examples of cycloalkoxy groups are cyclopropoxy, cyclobutoxy, cyclopentoxy, cyclohexyloxy, cycloheptyloxy and cyclooctyloxy.

### Thioalkyl

Thioalkyl as used herein refers to the group —alkyl, wherein alkyl is as defined above. Examples of thioalkyl groups are thiomethyl, thioethyl, n-thiopropyl, isothiopropyl, n-thiobutyl, isothiobutyl, sec-thiobutyl, t-thiobutyl, n-thiopentyl, isothiopentyl, neo-thiopentyl, n-thiohexyl, and isothiohexyl.

Sulfoxoalkyl

Sulfoxoalkyl as used herein refers to the group —S(O)-alkyl, wherein alkyl is as defined above.

### Sulfonoalkyl

Sulfonoalkyl as used herein refers to a the group —S(O)₂-alkyl wherein alkyl is as defined above.

### Halogen

Halogen, halide or halo-refers to iodine, bromine, chlorine and fluorine.

### Haloalkyl

Haloalkyl as used herein refers to an alkyl group as defined above wherein at least one hydrogen atom has been replaced with a halogen atom as defined above. Examples of haloalkyl groups include chloromethyl, dichloromethyl, trichloromethyl, fluoromethyl, difluoromethyl and trifluoromethyl. Preferred haloalkyl groups are fluoroalkyl groups (i.e. haloalkyl groups containing fluorine as the only halogen). More highly preferred haloalkyl groups are perfluoroalkyl groups, i.e. alkyl groups wherein all the hydrogen atoms are replaced with fluorine atoms.

### Aryl

As used herein, “aryl” refers to an unsaturated aromatic carbocyclic group of from 6 to 10 carbon atoms having a single ring (e.g., phenyl) or multiple condensed (fused) rings (e.g., naphthyl). Preferred aryl groups include phenyl, naphthyl and the like.

### Prodrugs

Also contemplated within the invention are prodrugs, that is compounds capable of undergoing metabolism to give compounds of formula (I) or (II) as defined above. Suitable prodrugs are N-oxides and compounds having a quaternary nitrogen.

### Leaving Group

The term “leaving group” as used herein refers to any moiety or atom that is susceptible to nucleophilic substitution or elimination. Typically, these are atoms or moieties that when removed by nucleophilic substitution or elimination are stable in anionic form. Examples of leaving groups useful in the present invention include alkyl- or arylsulphonate groups such as tosylate, brosylate, mesylate or nosylate, or halides such as fluoride, chloride, bromide, or iodide.

### Multiply Occurring Groups

When any variable (e.g. aryl, heterocycle, R⁷ etc.) occurs more than one time in any compound of the invention, its definition at each occurrence is independent of its definition at every other occurrence.

### Therapy

The compounds of the invention have therapeutic properties. They are expected to be useful in the treatment of proliferative diseases such as cancer, autoimmune diseases, viral diseases, fungal diseases, neurological/neurodegenarative diseases, arthritis, inflammation, alopecia, and cardiovascular disease.

### Cancer

More specifically, the compounds of the invention can be useful in the treatment of a variety of cancers, including (but not limited to) the following: carcinoma, including that of the bladder, breast, colon, kidney, liver, lung, including small cell lung cancer, esophagus, gall bladder, ovary, pancreas, stomach, cervix, thyroid, prostate, and skin, including squamous cell carcinoma; hematopoietic tumors of lymphoid lineage, including leukemia, acutelymphocytic leukemia, acutelymphoblasticleukemia, B-cell lymphoma, T-cell lymphoma, Hodgkins lymphoma, non-Hodgkins lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including melanoma, seminoma, teratocarcinoma, osteosarcoma, xenoderoma pigmentosum, keratoctanthoma, thyroid follicular cancer and Kaposi's sarcoma.

### Apoptosis

Compounds of the invention may induce, inhibit or modulate apoptosis. The apoptotic response is aberrant in a variety of human diseases. Compounds of the invention, as modulators of apoptosis, will be useful in the treatment of cancer (including but not limited to those types mentioned hereinabove), viral infections (including but not limited to herpevirus, poxvirus, Epstein-Barr virus, Sindbis virus and adenovirus), prevention of AIDS development in HIV-infected individuals, autoimmune diseases (including but not limited to systemic lupus, erythematosus, autoimmune mediated glomerulonephritis, rheumatoid arthritis, psoriasis, inflammatory bowel disease, and autoimmune diabetes mellitus), neurodegenerative disorders (including but not limited to Alzheimer's disease, AIDS-related dementia, Parkinson's disease, amyotrophic lateral sclerosis, retinitis pigmentosa, spinal muscular atrophy and cerebellar degeneration), myelodysplastic syndromes, plastic anemia, ischemic injury associated with myocardial infarctions, stroke and reperfusion injury, arrhythmia, atherosclerosis, toxin-induced or alcohol related liver diseases, hematological diseases (including but not limited to chronic anemia and plastic anemia), degenerative diseases of the musculoskeletal system (including but not limited to osteoporosis and arthritis)-aspirin-sensitive rhinosinusitis, cystic fibrosis, multiple sclerosis, kidney diseases and cancer pain.

### Pharmaceutically Acceptable Salts

Pharmaceutically acceptable salts can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids when a compound of this invention contains a basic moiety. Salts may also be formed from organic and inorganic bases, preferably alkali metal salts, for example, sodium, lithium, or potassium, when a compound of this invention contains an acidic moiety.

### Asymmetry

The compounds of this invention may contain an asymmetric carbon atom and some of the compounds of this invention may contain one or more asymmetric centers and may thus give rise to optical isomers and diastereomers. While shown without respect to stereochemistry, the present invention includes such optical isomers and diastereomers; as well as the racemic and resolved, enantiomerically pure R and S stereoisomers; as well as other mixtures of the R and S stereoisomers and pharmaceutical acceptable salts thereof. It is recognized that one optical isomer, including diastereomer and enantiomer, or stereoisomer may have favorable properties over the other. Thus when disclosing and claiming the invention, when one racemic mixture is disclosed, it is clearly contemplated that both optical isomers, including diastereomers and enantiomers, or stereoisomers substantially free of the other are disclosed and claimed as well.

### Pharmaceutical Composition

The present invention accordingly provides a pharmaceutical composition which comprises a compound of this invention in combination or association with a pharmaceutical acceptable carrier. In particular, the present invention provides a pharmaceutical composition which comprises an effective amount of compound of this invention and a pharmaceutical acceptable carrier.

The compositions are preferably adapted for oral administration. However, they may be adapted for other modes of administration, for example, parenteral administration for patients.

In order to obtain consistency of administration, it is preferred that a composition of the invention is in the form of a unit dose. Suitable unit dose forms include tablets, capsules, and powders in sachets or vials. Such unit dose forms may contain from 0.1 to 100 mg of a compound of the invention. The compounds of the present invention can be administered orally at a dose range of about 0.01 to 100 mg per kg.

Such composition may be administered from 1 to 6 times a day, more usually from 1 to 4 times a day.

The compositions of the invention may be formulated with conventional excipients, such as fillers, a disintegrating agent, a binder, a lubricant, a flavouring agent, and the like. They are formulated in conventional manner.

### Processes of the Invention

The compounds of the present invention can for example be prepared according to the following reaction schemes or modification thereof using readily available starting materials, reagents and conventional synthetic procedures. It is also possible to make use of variants of these process steps, which in themselves are known to and well within the preparatory skill of the medicinal chemist. In the following reaction schemes R¹, R², R³, R⁴, and Cy¹ are as defined above.

By way of example, compounds of general formula (N) above may conveniently prepared as follows.

Treatment of nitrile (X) with potassium t-butoxide and ethyl formate gives intermediate enol (XI), which upon treatment with hydrazine gives substituted 3-aminopyrazole (XII) (scheme 1).

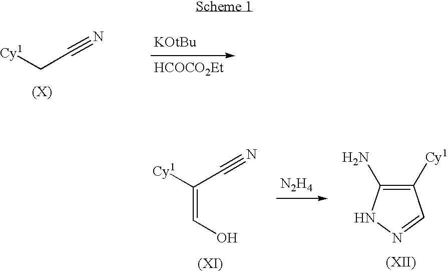

Keto ester (XV) is suitably prepared by treatment of ester (XIII) with strong base (e.g. lithium diisopropylamide) followed by acid chloride (XIV) (scheme 2).

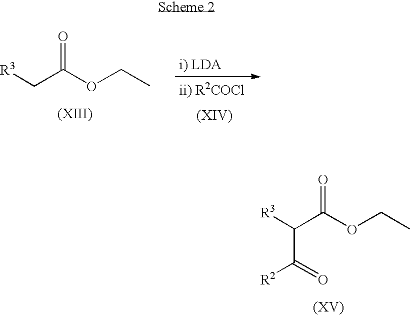

Reaction of keto ester (XV) with 3-aminopyrazole (XII) gives pyridone (XVI). This is suitably converted to the corresponding chloride (XVII) by treatment with phosphoryl chloride. Reaction with amine (XVIII) gives the desired product (scheme 3), which may subsequently be converted to a pharmaceutically acceptable salt form if desired.

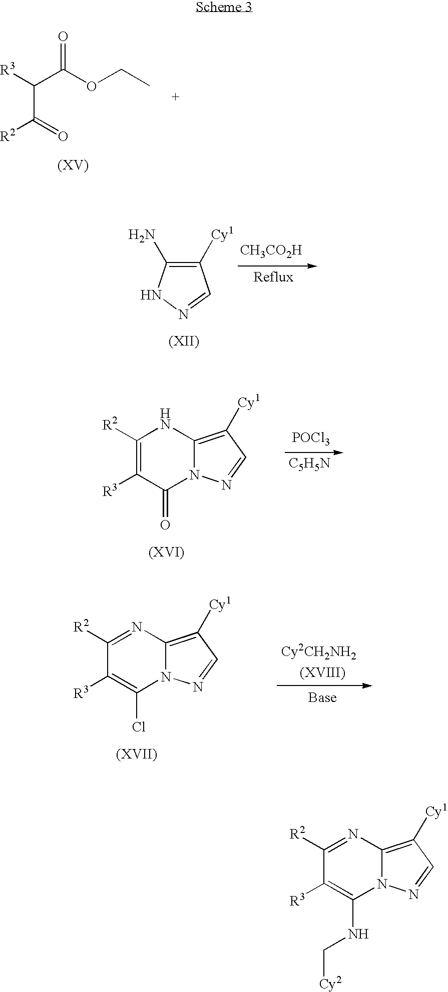

Alternatively, the compounds of the invention may be prepared by palladium-catalysed cross coupling of substituted 3 bromopyrazolo[1,5a]pyrimidines (XIX) with boronic acids (XX) to give 3-pyrazolo[1,5a]pyrimidines (I) or (II) (scheme 4).

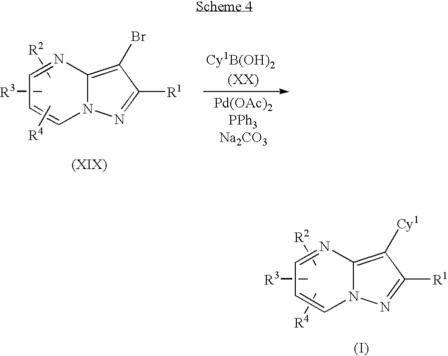

It will be apparent to those skilled in the art that sensitive functional groups may need to be protected and deprotected during synthesis of a compound of the invention. This may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

The present invention will now be described in further detail in the following examples.

### EXAMPLES

### Preparation of [3-(4-methoxyphenyl)pyrazol[1,5-a]pyrimidin-7-yl]pyridin-2-ylmethylamine

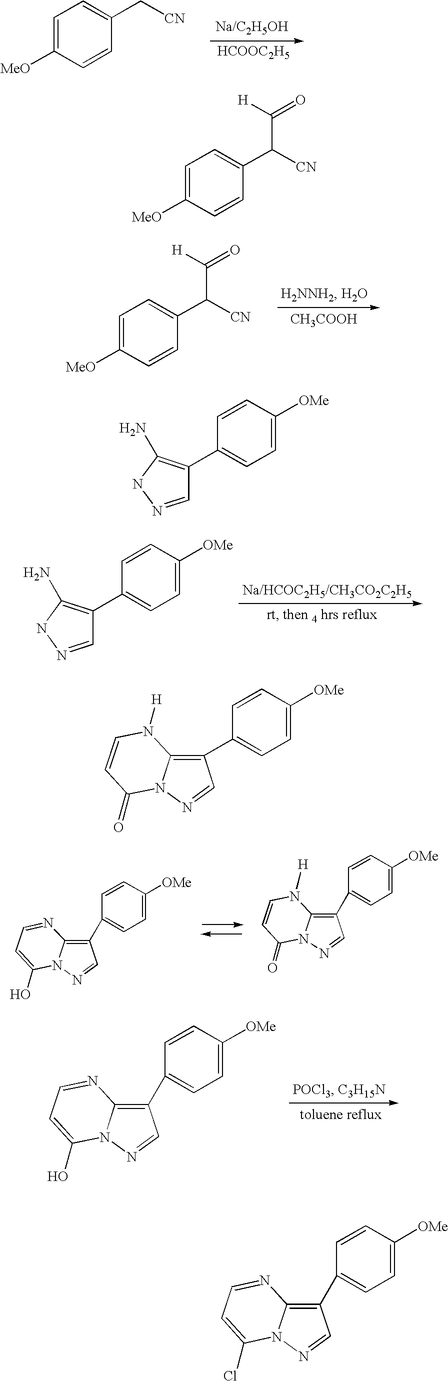

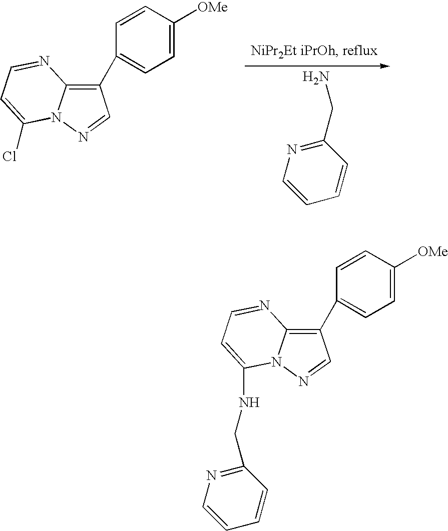

### 3-Oxo-2-(4-methoxyphenyl)propionitrile

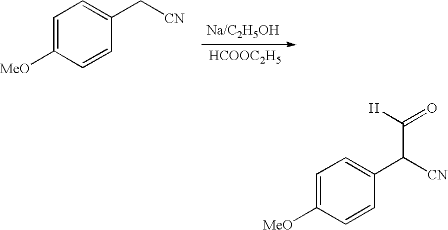

Raw Materials

| | |
|---|---|
| Ethyl formate 97% | Aldrich Cat. 11,268-2 |
| Sodium | Aldrich 282057-100G |
| Ethanol | Sigma-Aldrich E7023-500 ml |
| 4-Methoxyphenylacetonitrile | Avocado A14317 |
| Acetic acid | BDH Laboratory supplies |
| Dichloromethane | Riedel-de Haen 24233 |
| Magnesium sulfate: | Alfa Aesar 33337 |
| Methanol: | Aldrich 34860 |
| Dichloromethane | Riedel-de Haen 24233 |

Quantities

| | | | |
|---|---|---|---|
| Sodium | 5.09 g, 0.221 mol | 1.3 | eq. |
| 4-Methoxyphenylacetonitrile | 25 g, 0.17 mol | 1 | eq |
| Ethanol | 215 ml | | |
| Ethylformate | 18.8 g, 0.255 mol | 1.5 | eq. |

### Experimental Procedure

In a three-necked round-bottomed flask (500 ml) equipped with a reflux condenser fitted with inlet/outlet Nitrogen. The third neck was used to introduce sodium.

Ethanol was dried prior to use (over Mg/iodine): Reflux for three hours and distil.

Ethanol (215 ml) was poured in the three neck bottomed flask.

Sodium was added portionwise while stirring.

When the sodium was dissolved (1 hour), 4-methoxyphenylacetonitrile followed by ethyl formate were added. The funnel was rinsed with ca 4 ml of absolute ethanol and added to the reaction mixture.

Heat switched on and the temperature increased gradually to reflux and kept at this temperature for five hours.

On cooling, the reaction mixture (white milky) was transferred to a 1 L one necked round-bottomed flask and methanol was used to rinse the flask (ca. 100 ml). Then it was evaporated at reduced pressure, leaving a solid residue.

The solid residue was partitioned between 330 ml of water and 300 ml of CH2Cl2. The pH of the aqueous layer was readjusted to 3-4 by addition dropwise of con.HCl. The organic layer was separated and the aqueous layer extracted with further 200 ml of CH2Cl2.

The combined organic layer washed with 150 ml of water and finally with 150 ml of brine and dried over MgSO4.

Filtration and evaporation of the filtrate provided 28.15 gr of off while product. TLC (5% methanol in CH₂Cl₂) showed one spot and no raw material. NMR confirmed the structure.

### 4-(4-Methoxyphenyl)-2H-pyrazol-3-ylamine

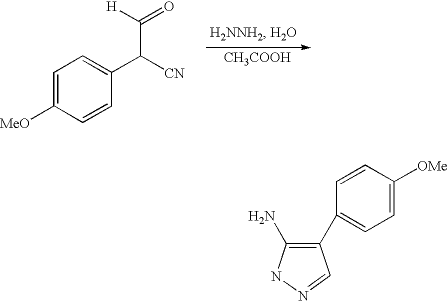

Raw Materials
4-methoxy-phenyl-3-oxopropionitrile (batch 0602100)

| | |
|---|---|
| Hydrazine monohydrate | Aldrich cat. 20,794-2 |
| AcOH | BDH |
| HCl | J. T. Baker 6081 |
| NH3 | J. T. Baker 6125 |
| Dichloromethane | Riedel-de Haen 24233 |

Quantities

| | | | |
|---|---|---|---|
| 3-Oxo-2 (4-methoxyphenyl) propionitrile | 28 g, 0.16 mol | 1 | eq. |
| Hydrazine monohydrate | 20, g, 0.4 mol | 2.5 | eq. |
| AcOH | 200 ml | | |

### Experimental Procedure

1 L one-necked round-bottomed flask equipped with magnetic stirrer and reflux condenser was charged with 3-Oxo-2 (4-methoxyphenyl)propionitrile (28 g, 0.16 moll), hydrazine monohydrate (20.02 g, 0.4 mol). Acetic acid (200 ml) was added with caution in three portions (steam was noticed).

The reaction mixture was stirred for 30 minutes before the heat switch on. Temperature kept at 96-98 degrees for five hours (while stirring).

The mixture was transferred to a 2 L one necked round-bottomed flask, 820 ml of water and 82 ml of conc. HCl were added and the flask was heated until reflux.

After six hours of reflux and on cooling down, ammonia hydroxide (35%) was added until pH=8-9, the mixture turned white and hot.

The mixture was filtered over ceramic filter. TLC (2% methanol in dichloromethane) of the solid showed no row material.

The material was suspended in 200 ml of ether and filtered. This operation was repeated three times (3×100 ml). The off-white solid was dried in the oven (70 degrees) until a constant weight

Yield: 23 gr.

The melting point of the compound was 178-180 degree C.

NMR confirmed the structure

### 3-(4-Methoxyphenyl)pyrazol[1,5-a]pyrimidin-7-one

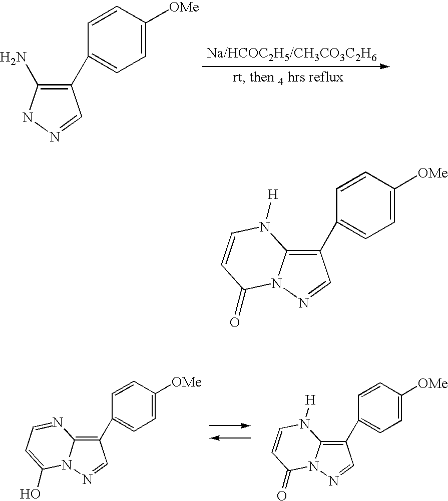

Raw Materials

| Na Metal | Aldrich 282057-100 G |
|---|---|
| Ethyl Formate | Aldrich 11,268-2 |
| Ethyl acetate | Aldrich 27227 |
| 4-Metoxy-phenyl-2H-Pyrazol-3-ylamine | |
| Ethanol absolute | Sigma-Aldrich E7023-500 ml |
| Toluene | Merck 108327 |
| Absolute Ether | Riedel-de Haen 24004 |
| HCl 36-38%. | T. Baker 6081 |

Quantities

| | |
|---|---|
| Na | −0.9 g, 39.1 mmol |
| Ethyl Formate | 2.5 ml, 46.25 mmol. |
| Ethyl acetate | 4.51 gr, 53.63 mmol |
| 4-Metoxyphenyl-2H-Pyrazol-3-ylamine | 3.32 g, 17.66 mmol |
| Toluene (anhydrous) | 15 ml |

### Experimental Procedure

100 ml one round-bottomed flask, equipped with a condenser and a magnetic stirrer was charged with sodium (0.9 g cut in small pieces) and covered with 2.5 ml of Toluene. Further Toluene (12 ml) was added followed by ethyl acetate, (4.1 g) and 0.3 ml of ethyl formate (using a 1 ml syringe). The reaction mixture was stirred at room temperature and a little heat (through a hot gun air) to the flask was applied to initiate the process. 30 minutes later, a yellow mixture appeared to be happening within the reaction. Further 1.5 ml of ethyl formate was added dropwise through the syringe and 1 hour later the remaining Ethyl formate (0.7 ml) was added.

The resultant mixture was stirred at RT for 72 hrs, in which a yellow suspension was formed and transferred into a 1 L one round-bottomed flask equipped with condenser and a magnetic stirrer. 500 ml of absolute ethanol was added, followed by 4-Methoxy-phenyl-2H-Pyrazol-3-ylamine (3.32 g). Further 50 ml of ethanol was used to rinse the flask containing the pyrazole.

The reaction mixture was stirred, heated gradually to reflux and kept at this temperature for six hours. After concentration of the reaction mixture under vacuum Pump (water pressure), the residue was dissolved in hot water (ca 50 ml) and the mixture was filtered quickly to remove the inorganic salt. The filtrate was then acidified to pH 1 with conc. HCl. On cooling, the solid was filtered, dried on air (2 hrs) then washed with ether (ca 10 ml) until the filtrate turned colourless to yellowish. Finally the solid was dried in the oven for 24 hrs at 70 degrees (till constant weight). (2.35 gr)

Nmr (DMSO-d6) demonstrated the formation of the desired material.

HPLC indicated purity of 90%.

### 7-Chloro-3-(4-methoxyphenyl)pyrazol[1,5-a]pyrimidine

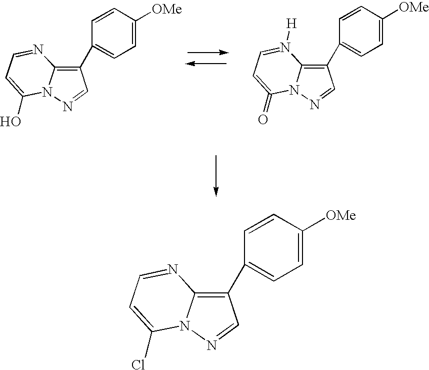

Raw Materials
3-(4-Methoxyphenyl)pyrazol[1,5-a]pyrimidin-7-one

| | |
|---|---|
| Phosphorus oxychloride | Aldrich cat.: 20,117-0 |
| N,N-Dimethylaniline | Aldrich cat.: 515124-1L |
| Magnesium sulfate: | Alfa Aesar 33337 |
| Silica gel: | Fluorochem LC 2025 |
| Dichloromethane | Riedel-de Haen 24233 |

Quantities
3-(4-Methoxy-phenyl)pyrazol[1,5-a]pyrimidin-
7-one 2.31 gr, 9.32 mmol.

| | | | | | | |
|---|---|---|---|---|---|---|
| Phosphorus oxychloride | 16 | ml | 26.32 | g | 0.000171 | m. mol |
| N,N-Dimethylaniiine | 1.52 | g | 1.6 | ml | 0.0000126 | m. mol |

### Experimental Procedure

To 2.31 g of 3-(4-Methoxyphenyl)pyrazol[1,5a]pyrimidin-7-one charged into a 50 ml round-bottomed flask was added 16 ml of phosphorus oxychloride and 1.6 ml of N,N-Dimethylaniline.

The solution was heated to 107 degree C. for 3 hrs then allowed to cool overnight while stirring. The reaction mixture was evaporated at reduced pressure.

Cold water (20 ml) was added to the residue in the flask (which cause some heat, cooled immediately in a beaker containing some ice). The aqueous residue was then poured into dichloromethane (80 ml) and more water was added (40 ml). The organic layer was separated and the aqueous layer extracted three times with dichloromethane (3×40 ml). The combined dichloromethane layers were dried over Magnesium sulfate.

Removal of the magnesium sulfate by filtration and concentration at reduce pressure. The residue was purified over silica gel column (using methanol/CH2Cl2 in various proportions.

### Purification Process:

80 gr of silica diluted first with 200 ml of dichloromethane, then with 600 ml of 5/95 (methanol/dichloromethane). Fractions of 25 ml were collected in test tubes: Tubes 13-15 had shown the desired material (after evaporation, we got 540 mg) (Rf=0.8 with 98/2=dichloromethane/methanol). Tubes 16-21 were contained the material contaminated with much polar impurities. So, a new purification on silica gel column on this contaminated material was carried on (ca lgr of material). Elution with dichloromethane (600 ml) and 200 ml of methanoVdichloromethane=1/99).

The total amount obtained from these two purifications: 955 mg. The NMR confirmed its structure and the purity by HPLC was above 92%.

### [3-(4-methoxyphenyl)pyrazol[1,5a]pyrimidin-7-yl]pyridin-2-ylmethylamine

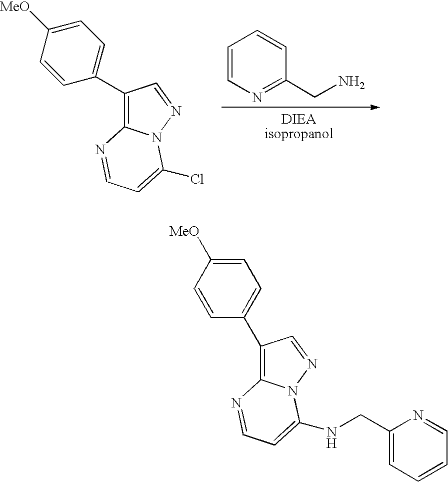

Raw Materials
7-Chloro-3-(4-methoxyphenyl)pyrazol[1,5-
a]pyrimidine: (Batch PR1303010)

| | |
|---|---|
| 2-Picolylamine | Aldrich A6, 520-4 |
| Diisopropylethylamine (DIEA) | Aldrich 49,621-9 |
| Isopropanol | Aldrich 32,047-1 |
| Dichloromethane | Riedel-de Haen 24233 |
| Water | Dionised water |
| Magnesium sulfate: | Alfa Aesar 33337 |
| Silica gel: | Fluorochem LC 2025 |

Quantities
7-Chloro-3-(4-methoxyphenyl)pyrazol[1,5-a]pyrimidine:
(Batch PR1 303010) 0.95 g, 3.66 mmol, 1 eq.

| | |
|---|---|
| 2-Picolylamine | 0.475 g, 4.393 mmol, 1.2 eq. |
| DIEA | 0.710 g, 5.494 mmol, 1.5 eq. |
| Isopropanol | 25 ml |

### Experimental Procedure

To the pyrimidine (0.95 gr) (Batch 1303010) in isopropanol (25 ml) was added DIEA 0.710 gr, followed by picolylmethylamine.

Heating gradually to 85 degrees over 45 min. and kept at this temperature for 15 hr while stirring.

Tlc (2% methanol in dichloromethane) showed a polar product at 0.25 with a very impurity close to the Rf of raw material (we demonstrated later by HPLC there was no raw materials.

The reaction mixture was evaporated to dryness. The yellow residue was partitioned between 200 ml of dichloromethane and 100 ml of water. The organic layer was separated and the aqueous layer extracted again with 40 ml of dichloromethane.

The combined organic layers were washed with 10% citric acid (50 ml), brine (50 ml) and finally dried (MgSO4).

After filtration and evaporation, we got 1.1 gr of the desired product (as a yellow solid) as confirmed by NMR HPLC: 94.85 purity) with no raw materials. It is identical to the sample provided by Eirx (same retention time and same UV absorption).

### Preparation of 7-Hydroxypyrazolo[1,5a]pyrimidine

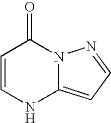

Sodium (30 wt. % dispersion in toluene, Acros 24326) (250 mL, 2.9 mol), toluene (1 L) and ethyl acetate (290 mL, 3 mol) were added to a 5 L three necked, round-bottomed flask. Ethyl formate (30 mL, 0.37 mmol) was added to the reaction mixture and the contents heated to 35-40° C. using a hot air gun. After initiation (2-16 h), ethyl formate (206 mL, 2.55 mol) was added dropwise to the suspension, taking care to ensure that the reaction temperature did not rise above 35° C. After stirring for 16 h at room temperature, a solution of 3-aminopyrazole (122 g, 1.47 mol) in ethanol (1 L) was added to the brown mixture and was refluxed for 4 h. The viscous, yellow suspension was allowed to cool and was concentrated in vacuo, the residue was dissolved in hot water (500 mL) and the contents acidified by the addition of conc. HCl (140 mL). The precipitate was collected by filtration and dried to yield the desired product, 7-hydroxypyrazolo[1,5-a]pyrimidine, as a white solid (152 g, 77%). ¹H NMR (250 MHz, DMSO) δ 12.37 (brs, 1H), 7.86 (d, J=7.3 Hz, 1H), 7.85 (d, J=2.0 Hz, 1H), 6.16 (d, J=2.0 Hz, 1H), 5.66 (d, J=7.3 Hz, 1H); ¹³C NMR (62.9 MHz, DMSO) δ 156.6, 142.6, 141.7, 139.4, 95.2, 88.9; MS (APCl) 136 [M+H]⁺.

### 7-Chloropyrazolo[1,5-a]pyrimidine

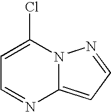

7-Hydroxypyrazolo[1,5-a]pyrimidine (60 g, 444 mmol), phosphorus oxychloride (108 mL, 1.15 mol) and dimethylaniline (10.8 mL, 89 mmol) were added to a 250 mL round-bottomed flask and the contents heated to 90° C. After 2.5 h, the reaction mixture was allowed to cool and was concentrated. The black residue was poured into a beaker containing crushed ice (600 mL) and the resulting solution kept cold using an ice bath. The aqueous solution was extracted with CH₂Cl₂ (3×150 mL) and the combined organic phase was washed with brine (100 mL). The organic phase was dried and concentrated to yield the desired crude product, 7-chloropyrazolo[1,5-a]pyrimidine, (44.2 g, 65%) as a red solid. As the crude product was sufficiently pure to use in the subsequent step, a small sample was purified by column chromatography (33-66% EtOAc/hexane) to yield an analytical sample as a white solid. HPLC 95%; ¹H NMR (250 MHz, CDCl₃) δ 8.41 (d, J=4.5 Hz, 1H), 8.25 (d, J=2.3 Hz, 1H), 6.99 (d, J=4.5 Hz, 1H), 6.84 (d, J=2.3 Hz, 1H); ¹³C NMR (62.9 MHz, CDCl₃) δ 150.0, 148.2, 145.4, 139.0, 108.0, 98.7; MS (APCl) 154 [M+H]⁺.

### 3-Bromo-7-chloropyrazolo[1,5-a]pyrimidine

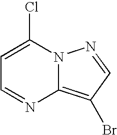

A solution of crude 7-chloropyrazolo[1,5-a]pyrimidine (44.2 g, 289 mmol) in dichloromethane (289 mL) was cooled to 0° C. using an ice bath. N-bromosuccinimide (51.4 g, 289 mmol) was added slowly to the solution that was stirred for 16 h at room temperature. The dark orange solution was diluted with dichloromethane (200 mL) and was washed with 10% potassium carbonate solution (3×150 mL) and brine (100 mL).

The organic phase was dried and concentrated to give a dark orange solid, which was triturated using MeOH to yield the desired product, 3-bromo-7-chloropyrazolo[1,5-a]pyrimidine (50.9 g, 76%) as a yellow solid. HPLC 96%; ¹H NMR (250 MHz, CDCl₃) δ 8.49 (d, J=5.2 Hz, 1H), 8.25 (s, 1H), 7.07 (d, J=5.2 Hz, 1H); ¹³C NMR (62.9 MHz, DMSO) δ 150.4, 145.9, 144.7, 138.5, 109.5, 84.7.

### General method for the amination of 3-bromo-7-chloropyrazolo[1,5-a]pyrimidine

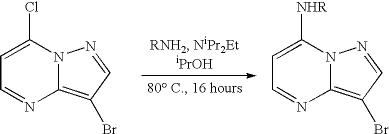

The amine (1.1 equivalents) and 1.5 equivalents of diisopropylethylamine in isopropanol at a total concentration of 1M were heated overnight at 80° C. The reaction mixture was concentrated, washed with water, filtered, and the resulting solid partitioned between ethyl acetate or dichloromethane and water. After a further water wash, and a citric acid wash with amines possessing no additional basic functionality, the organic layer was dried then concentrated to give the crude 7-substituted amino-3-bromopyrazolo[1,5a]pyrimidine in good yield. The purity of this material was sufficient for use in the next stage cross-coupling reaction. As Example 1, the use of 2-aminomethylpyidine gave 7-aminomethyl-3-bromopyrazolo[1,5a]pyrimidine in 91% yield and 99% purity (MS: m/z 306/304 [M+H]⁺).

### 3-Bromo-7-(2-thiophenemethylamino)pyrazolo[1,5-a]pyrimidine

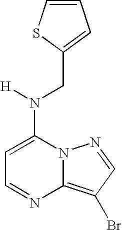

To a solution of 3-bromo-7-chloropyrazolo[1,5-a]pyrimidine (9.63 g, 41.5 mmol) and diisopropylethylamine (10.9 mL, 62.3 mmol) in 2-propanol (42 mL) was added 2-(aminomethyl)thiophene (5.6 g, 49.8 mmol). After heating for 16 h at 80° C., the reaction mixture was concentrated and the residue partitioned between dichloromethane and water. After separation, the organic phase was washed with 10% citric acid and brine, dried and concentrated to yield the desired 3-bromo-7-(2-s thiophenemethylamino)pyrazolo[1,5-a]pyrimidine (11.49 g, 92%) as a brown solid. HPLC 99%; ¹H NMR (250 MHz, CDCl₃) δ 8.88 (t, J=6.4 Hz, 1H), 8.25 (s, 1H), 8.18 (d, J=5.3 Hz, 1H), 7.38 (dd, J=1.3, 5.1 Hz, 1H), 7.13 (dd, J=1.3, 3.4 Hz, 1H), 6.95 (dd, J=3.4, 5.1 Hz, 1H), 6.29 (d, J=5.3 Hz, 1H), 4.80 (d, J=6.4 Hz, 2H); ¹³C NMR (62.9 MHz, CDCl₃) δ 150.5, 146.7, 145.5, 143.1, 140.8, 126.8, 126.2, 125.4, 86.5, 81.0, 40.0; MS (APCl) 311/309 [M+H]⁺.

### General method for BOC protection of 7-substituted amino-3-bromopyrazolo[1,5a]pyrimidines

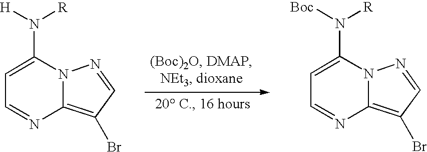

The 7-substituted amino-3-bromopyrazolo[1,5a]pyrimidine (1 equivalent) was stirred overnight at room temperature with 1.5 equiv. (Boc)₂O, 0.1 equiv.-DMAP, 1.5 equiv. NEt₃ in dioxane. The reaction mixture was concentrated, the crude product partitioned between ethyl acetate or dichloromethane and water, the organic layer dried, passed through a pad of silica, and concentrated to give the 7-substituted Boc-amino-3-bromopyrazolo[1,5a]pyrimidine in good yield.:

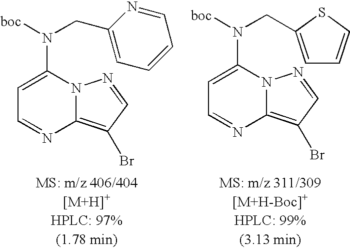

### General method for Suzuki cross-coupling of 7-substituted Boc-amino-3-bromopyrazolo[1,5a]pyrimidines

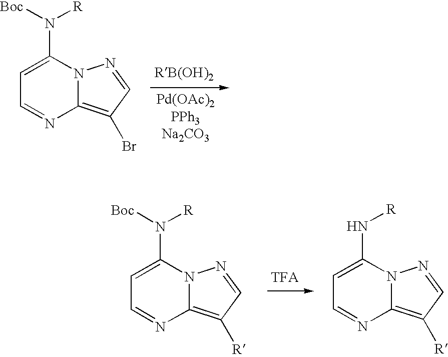

To a 10 mL screw-capped glass tube was added a solution of the boronic acid (0.33 mmol) in DMF (0.6 mL), a solution of the 7-substituted Boc-amino-3-bromopyrazolo[1,5a]pyrimidine (0.3 mmol) in DMF (0.8 mL) and a solution of sodium carbonate (0.6 mmol) in water (0.4 mL). The reaction tube was transferred to the glove box and after the atmosphere had equilibrated, a suspension of Pd(OAc)₂ (150 mol) and PPh₃ (450 mol) in dioxane (0.2 mL) was added. The tube was capped and heated for 16 h at 80° C. After allowing to cool, the reaction was filtered into a 48 well plate and was concentrated to dryness. The residue was dissolved in 80% TFA/CH₂Cl₂ (2 mL) and was allowed to stand for a further 2 h. The solution was again concentrated and was redissolved in DMSO (2-mL). The crude solution was purified by preparative HPLC and the product was isolated after lyophilisation.

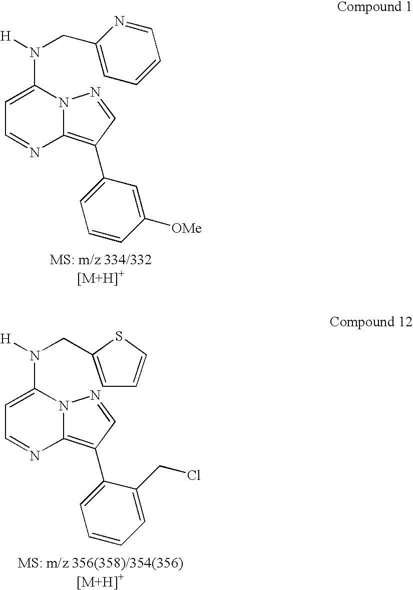

Compounds 2 to 11 and 13 are also prepared by this general method.

### Preparation of Compound 14

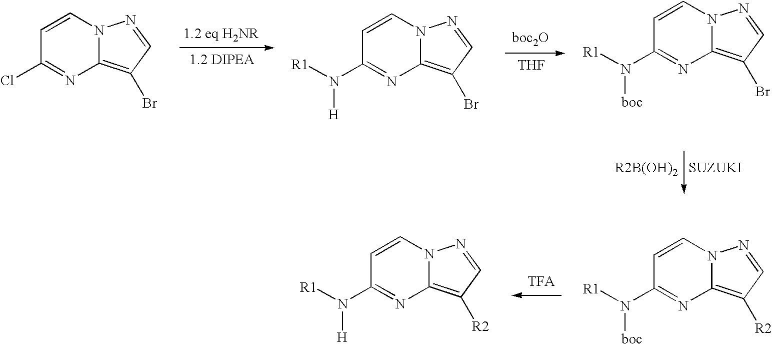

The scaffold (A) was sourced commercially from Butt Park Ltd., Bath, UK. The synthesis of (14) was carried out exactly as described for examples (1)-13) using the appropriate scaffold, amine and boronic acid, giving Compound (14) in good yield, MS: m/z 324(326)/322(324) [M+H]+

The ability of the compounds to act as antineoplastic agents was established by the following experimental procedures:

### Example 2

### Generation of TF-1 that are Engineered to Overexpress the FRAT2 Gene

This example describes how the erythroleukemic TF-1 cell line was engineered to express the FRAT2 gene linked to the reporter Green Fluorescent Protein (GFP) gene via an Internal Ribosomal Entry Site (IRES). It also describes how a monoclonal FRAT2 cell line was isolated.

### Preparation of the FRAT2 Expression Construct:

This example describes the cloning of the full length coding sequence (cds) of FRAT2 (GenBank Accession No. BC020165) using FRAT2 specific amplification primers and a TOPO-TA cloning kit from Invitrogen. It further describes how the FRAT2 eds is incorporated into a construct such that it is linked to a gene encoding the human recombinant Green Fluorescent Protein (hrGFP) using an internal ribosomal entry site. Primers FRAT2 forward (5′-ccaagcttGGACGGGGGGCCATGCCG-3′) and FRAT2 reverse (5′-GGCCTGCGTCAGAGCAAGGAGC-3′) are used to amplify a 721 bp DNA sequence containing the 702 bp eds of FRAT2 from HL60 cDMA using Qiagen Taq (#201207). This DNA fragment is gel isolated using Qiagen Gel Extraction Kit (#28706). The DNA fragment is then incubated with Taq and dATPs for 5 min. at 72° C. to add on 3′ A-overhangs on the amplified DNA which may have been lost during gel extraction, but which are necessary for TOPO-TA cloning.

The DNA fragment is “TOPO” cloned into Invitrogen's pcDNA3.1/V5-His TOPO TA expression vector according to the manufacturers instructions, and transformed into Top10 E coli cells. Transformed cells are plated on ampicillin containing Agar plates for selection. Colonies are screened for the FRAT2 insert using 17 forward (5′-TAATACGACTCACTATAGGG-3′) and BGH reverse (5′-TAGAAGGCACGTCGAGG-3′) primers which yields a 987 bp product if FRAT2 has been successfully cloned or a 266 bp product in the absence of any insert. Colonies are screened for inserts in the sense orientation using T7 forward and FRAT2 reverse primers and in the antisense orientation using T7 forward and FRAT2 forward primers.

Glycerol stocks of FRAT2-containing clones, pcDNA3.1/V5-His TOPO-FRAT2, are prepared and stored at −70° C. Three different clones are sequenced and a clone containing no mutations is selected for further use. A large-scale preparation of the selected plasmid is purified using Qiagen's Plasmid Mid Kit (#112145). The concentration of the plasmid is adjusted to 1 mg/ml for convenience during subsequent manipulations.

For expression in mammalian cells the FRAT2 gene is cloned into a modified pMSCVhyg retroviral vector (Clontech #K1062-1) using the GATEWAY System (Invitrogen), as detailed below.

The IRES and hrGFP genes from pIREShrGFP-1a (Stratagene #240031) (position 710-2446) is cloned into the Xho1 and Hpa1 sites of pMSCV by directional cloning, resulting in a construct called pMSIRG. This allows the level of expression of a gene of interest to be monitored due to the expression of hrGFP on the same transcript. The blunt ended attR cassette (reading frame B), from Invitrogen's GATEWAY Conversion System (#11828-19), is ligated into pMSIRG that is digested with Xho1 and blunt-ended using Klenow. Recombinant constructs are selected in which the attR cassette is in the sense orientation, namely pMSIRGattRS.

In order for the FRAT2 gene to be cloned into the pMSIRGattRS expression vector, it is first cloned into the pDONR 201 vector (Invitrogen #1179-014) as follows:

The FRAT2 gene is amplified from pcDNA3.1/V5-His TOPO-FRAT2 plasmids using attB-linked T7 and BGH primers, i.e. T7-AttB1 forward primer (5′-GGGGACAAGTTTGTACAAAAAAGCAGGCTTAACGACTCACTATAGGG-3′) and BGH-attB2 reverse primer (5′-GGGGACCACTTTGTACAAGAAAGCTGGGTTAGAAGGCACAGTCGAGG-3′). The resulting 1045 bp product is gel extracted (using Qiagen Gel Extraction kit #28706) and cloned into pDONR201 via a GATEWAY BP cloning reaction according to Invitrogen's instructions, to generate pDONR201-FRAT2. Recombinant clones are selected on kanamycin-containing agar plates. Recombinant plasmids are purified using Qiagen QIAprep Spin Miniprep Kit (#27104) and the presence of the insert confirmed by restriction analysis.

A GATEWAY LR cloning reaction is performed using pDONR201-FRAT2 and pMSIRGattRS according to the GATEWAY Technology Instruction manual to generate pMSIRGattRS-FRAT2. Recombinant clones are selected on amplicillin-containing agar plates. Recombinant plasmids are purified using Qiagen QIAprep Spin Miniprep Kit (#27104) and the presence of the insert confirmed by restriction analysis. A large-scale preparation of a selected clone is purified using Qiagen's Plasmid Mid Kit (#112145). The concentration of the plasmid is adjusted to 1 mg/ml for convenience subsequent viral transductions into mammalian cells.

Transduction of FRAT2 Expression Construct into Mammalian Cells

This example describes how the Erytholeukemic TF-1 cell line are infected with retrovirus incorporating the FRAT2 gene, thus allowing for expression of said gene.

pMSIRGattRS-FRAT2 containing ecotropic retrovirus is generated by transfecting a mono-layer of 70-80% confluent Phoenix-ECO PCL (Packaging Cell Line) cells (licensed from Stanford University) with 10 μg of pMSIRGattRS-FRAT2 plasmid in a T25 flask in media containing DMEM and freshly prepared 25 μM chloroquine diphosphate (Sigma #C6628) using the Clontech CalPhos mammalian transfection kit (#PT3025-1) according to the manufacturers instructions. Cells are incubated at 37° C. for 18 h after which the transfection medium is replaced with fresh serum-free RPMI-1640 medium. Cells are incubated at 32° C. during which time virus is released into the supernatant. After 48 h the supernatant is harvested by centrifugation and filtered through a 0.45 μM filter and used immediately.

Logarithmically growing C9M TF1 cells that have been modified to constitutively express the murine retrovirus receptor, mCAT, and thus are amenable to transduction with murine-retrovirus, are used for the infection with recombinant virus and expression of exogenous. FRAT2. The cells are spun down and re-suspended at a concentration of 1×10⁵ cells/ml in normal growth medium containing 2 ng/ml GM-CSF. Freshly prepared polybrene [Hexadimethrine bromide] (Sigma #H9268) is added to the viral suspension to a concentration of 8 g/ml. The virus/poylbrene cocktail is added to the cells in appropriate culture vessel and these are incubated at 37° C. for 18 h after which the virus-containing supernatant is replaced with fresh RPMI-1640 growth medium, 2 ng/ml GM-CSF and maintained at 37° C. at a final cell density of 2×10⁵ cells/ml.

Stably-transduced C9M-TF1 populations are selected and non-transduced populations are eliminated by adding 500-600 μg/ml hygromycin at 48 h post infection. Selected cells are monitored for cytopathic effects and re-fed with fresh selection medium every 34 days. Following the death of all mock-transduced cells, the selected population of 100% transduced cell are expanded for expression analysis/assay/freezing etc.

### Generation of Monoclonal FRAT2 Cells

This example describes a procedure whereby monoclonal FRAT2 cells are isolated. Monoclonal cell lines are preferred in the current series of experiments due to their uniform response.

A dilute cell suspension of polyclonal FRAT2 cells is prepared at 1.5×10³ cells/ml. A forceps is used to partially submerge a 200 μl Gilson tip in the dilute cell suspension until the tip becomes filled with medium. The point of the tip is gently pressed against the bottom of each well of a row of a 96-well plate such that a tiny droplet forms in the centre of the well. The droplets are viewed under the microscope (10×) to determine how many, if any, contain a single cell per droplet. Each droplet is independently screened by two individuals, to verify which wells contain a single cell. The process is repeated to generate several wells having a single cell per well. Each well marked was filled with 100 μl of culture medium (RPMI 1640+20% FCS+GMCSF). Plates are incubated at 37° C. in 5% CO₂ for two weeks. Plates are checked periodically to ensure medium has not been lost through evaporation. Wells are replenished with fresh medium if necessary. Cells are grown to confluency and then expanded into larger cell culture vessels. At all times individual monoclonals are treated at as different cell lines. The monoclonal line used in these experiments is referred to as FRAT2 C9M-TF1 cells.

### Example 3

### Quantitative PCR Analysis of FRAT2 Cells

This example describes “real time” quantitative PCR (QPCR) analysis performed on TF-1 cells that have been engineered to express the FRAT2 cds. With the ability to measure the PCR products as they are accumulating, or in “real time”, it is possible to measure the amount of PCR product at a point in which the reaction is still in the exponential range. It is only during this exponential phase of the PCR reaction that it is possible to extrapolate back to determine the starting amount of template. During the exponential phase in real-time PCR experiments a fluorescence signal threshold is determined at which point all samples can be compared. This threshold is calculated as a function of the amount of background fluorescence and is plotted at a point in which the signal generated from a sample is significantly greater than background fluorescence. Therefore, the fractional number of PCR cycles required to generate enough fluorescent signal to reach this threshold is defined as the cycle threshold, or Ct. These Ct values are directly proportionate to the amount of starting template and are the basis for calculating mRNA expression levels or DNA copy number measurements.

Levels of FRAT2 mRNA in Engineered-in FRAT2 C9M-TF1 Cells:

This example describes QPCR analysis of C9M-TF-1 cells that have been engineered to express the FRAT2 cds and where the expression of messenger RNA for FRAT2 is measured.

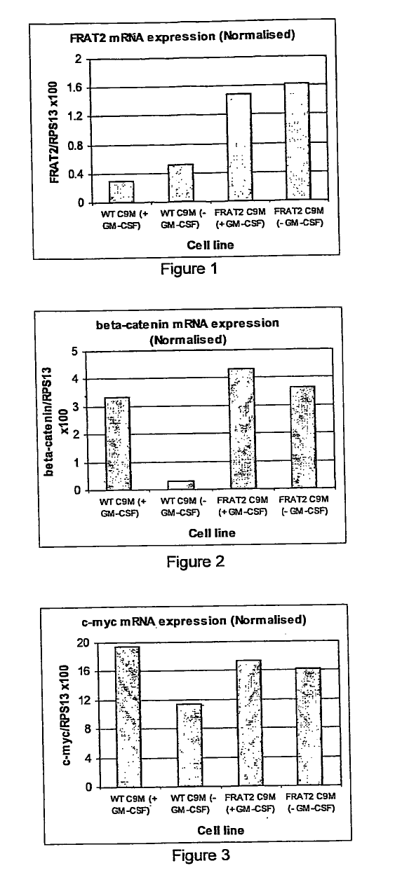

Logarithmically growing WT C9M-TF1 cells and recombinant C9M-TF1 cells transduced with pMSIRGattRS-FRAT2 are harvested by centrifugation 24 h post splitting in the presence or absence of GM-CSF. The RNA is purified using Qiagen RNeasy Kits (#74104) and Qiashredders (#79654) according to manufacturers protocol. cDNA is synthesized from this RNA using Superscript II Reverse Transcriptase from Invitrogen (#18064-014) according to the manufacturers instructions. Quantitative real time PCR is performed on the cDNA samples using QuantiTect SYPR Green Kit (according to the manufacturers instructions) and FRAT2 Quantitative PCR primers (QFRAT2 forward: 5′-AGCGCCGATGGACCCAAGC-3′; QFRAT2 reverse: 5′-AGGGCAATGCGGTCAGGTCC-3′) on a DNA Engine Opticon system from MJ Research (PTC200DNA Engine Cycler; CFD-3200 Opticon Detector). The expression level of FRAT2 is normalized with the expression level of the housekeeping gene RPS13. FIG. 1 shows an example of such analysis.

Quantitative Real-time PCR shows that increased levels of FRAT2 mRNA are detected in FRAT2 expressing cells compared to wild type (WT) C9M-TF1 cells whether they are grown in the presence or absence of GM-CSF. Exogenous expression of FRAT2 results in an almost 5 fold increase in mRNA levels in cells grown in the presence of GM-CSF, and 3 fold increase in cells grown in the absence of GM-CSF.

FIG. 1 shows levels of β-catenin in FRAT2 C9M-TF1 cells compared with wild type C9M-TF1 following GMCSF withdrawal.

This example describes the effect of withdrawing GM-CSF from C9M-TF-1 cells on levels of β-catenin, and how expression of FRAT2, as exemplified in FRAT2 C9M-TF1 cells, is able to maintain levels of the catenin protein.

Either wild type C9M-TF1 cells or FRAT2-C9M-TF1 are cultured in the presence or absence of GMCSF for 24 h after which time RNA is isolated. The RNA is purified using Qiagen RNeasy Kits (#74104) and Qiashredders (#79654) according to manufacturers protocol. cDNA is synthesized from this RNA using Superscript II Reverse Transcriptase from Invitrogen (#18064-014) according to the manufacturers instructions. Quantitative real time PCR is performed on the cDNA samples using QuantiTect SYPR Green Kit (according to the manufacturers instructions) and the quantitative PCR primers as indicated below are used with a DNA Engine Opticon system from MJ Research (PTC200DMA Engine Cycler, CFD-3200 Opticon Detector). The expression level of all genes is normalized with the expression level of the housekeeping gene RPS13. Q-PCR analysis was performed as described above using Qbeta-catenin forward (5′-ATCCCACTAATGTCCAGCGTTEG-3′) and Qbeta-catenin reverse (5′TGCAAGGTCCCAGCGGTACA-3′).

When GM-CSF is withdrawn from the factor dependent C9M-TF-1 cells for 24 h, levels of β-catenin is seen to decrease dramatically. However, this decrease is not observed in FRAT2 C9M-TF1 cells, as can be seen in FIG. 2, thus demonstrating that expression of FRAT2 can stabilize the transcription of β-catenin over a 24 h period.

FIG. 2 shows that withdrawal of GM-CSF for 24 h causes a dramatic decrease in the level of β-catenin expression; however overexpression of FRAT2 is able to rescue this decrease.

### Validation of Cells by Q-PCR of Beta-Catenin and Beta-Catenin Regulated Genes.

This example describes QPCR analysis on FRAT2-C9M-TF1 cells where the genes examined are known to be regulated by β-catenin. FRAT2 in known as a positive regulator of the wnt signaling pathway by displacing GSK3p from the axin/beta-catenin complex, thereby facilitating the translocation of beta-catenin to the nucleus and the transcriptional activation of several downstream genes including c-myc, cyclin D1 and con.

Either wild type C9M-TF1 cells or FRAT2-C9M-TF1 are cultured in the presence or absence of GMCSF for 24 h after which time RNA is isolated. The RNA is purified using Qiagen RNeasy Kits (#74104) and Qiashredders (#79654) according to manufacturers protocol. cDNA is synthesized from this RNA using Superscript II Reverse Transcriptase from Invitrogen (#18064-014) according to the manufacturers instructions. Quantitative real time PCR is performed on the cDNA samples using Quanti-Tect SYPR Green Kit (according to the manufacturers instructions) and the quantitative PCR primers as indicated below are used with a DNA Engine Opticon system from MJ Research (PTC200DNA Engine Cycler; CFD-3200 Opticon Detector). The expression level of all genes is normalized with the expression level of the housekeeping gene RPS13.

The Q-PCR specific primer pairs Qc-myc forward 5′-CCGCCCCTGTCCCCTAGC-3′ and Qc-myc reverse 5′-TCCGGGTCGCAGATGAAACTC-3′; Qcyclin D1 forward 5′-CGCGCCCTCGGTGTCCTACT-3′ and QcyclinD1 reverse 5′-GCGGCCAGGTTCCACTTGAG-3′ reverse; Qcox2 forward 5′-TGCCTGATGATTGCCCGACTC-3′ and Qcox2 reverse 5′-TGGCCCTCGCTTATGATCTGTCT-3′.

The withdrawal of GM-CSF from C9M-TF-1 cells results in a drop in the expression level of the three genes examined; however the decrease is not so significant in FRAT2 C9M-TF1 cells, thus demonstrating that cells expressing FRAT2 can effect the transcriptional levels of β-catenin regulated genes.

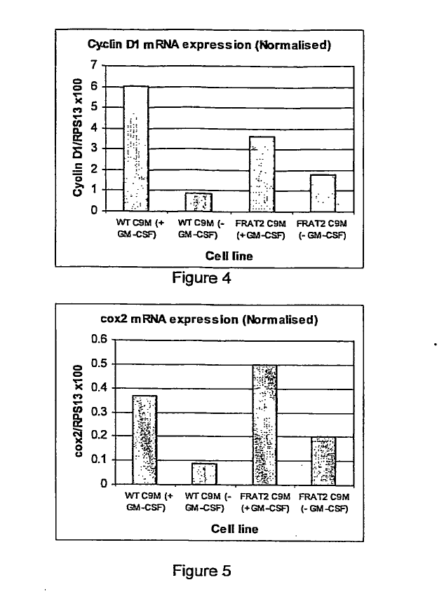

FIGS. 3, 4 and 5: withdrawal of GM-CSF can affect expression of the β-catenin regulated genes c-myc, Cyclin D1 and Cox2. However overexpression of FRAT2 reduces the decrease in expression, thus demonstrating that FRAT2 can positively affect β-catenin regulated genes.

### Example 4

### Demonstration that FRAT2 C9M-TF1 are More Resistant to Apoptosis than Wild Type C9M-TF-1

This example describes the assays used to determine whether FRAT2 C9M-TF1 engineered cells are more resistant to apoptosis than their wild type C9M-TF1 cells.

### Forward Scatter/Side Scatter (FSC/SSC) Analysis of Apoptosis

FSC/SSC analysis utilised in this example represents an apoptosis assay that can distinguish accurately between cells that have undergone apoptosis or necrosis from viable cells. This is based on the characteristic changes in cell size and granularity associated with viable, apoptotic and necrotic morphology. To ascertain the characteristic cell size and granularity of a cell population, FRAT2 C9M-TF1 and C9M-TF-1 cells are analysed utilising the flow cytometer. The position of the cell population on the FSC and SSC scale are noted by isolating the population with a gate. There is no difference in FSC/SSC parameters for either FRAT2 C9M-TF1 or C9M-TF1 in healthy cultures. The effect of removing GMCSF on cell viability is ascertained by observing the movement of the cell population from the viable gate previously recorded. Necrotic cells increase in size before disintegrating and therefore are noted to shift up and to the right of the viable cell population initially while next shifting completely into the debris section of the FSC/SSC plot (bottom left) when disintegrated. Apoptotic cells however simply move to the left of the viable population gradually reducing in size, and then shift gradually down the SSC scale as the granularity of the cells increases in late stage apoptosis.

Therefore due to the cell size and granularity changes associated with apoptosis, the changes in FSC/SSC parameters can be used to identify cells undergoing apoptosis post GMCSF withdrawal. Cells are cultured for 72 h with or without GMCSF (2 ng/ml) after which they are harvested, and acquired immediately by a FacsCalibre (Bectori Dickenson). Forward and Side Scatter parameters are assessed using Cell Quest software. The FSC/SSC parameters of the cell population cultured in the presence of GMCSF is compared to the FSC/SSC parameters of the cells cultured in the absence of GMCSF. The percentage of cells remaining within the viable cell gate post 72 h GMCSF starvation is ascertained for both FRAT2 C9M-TF1 or C9M-TF1 populations.

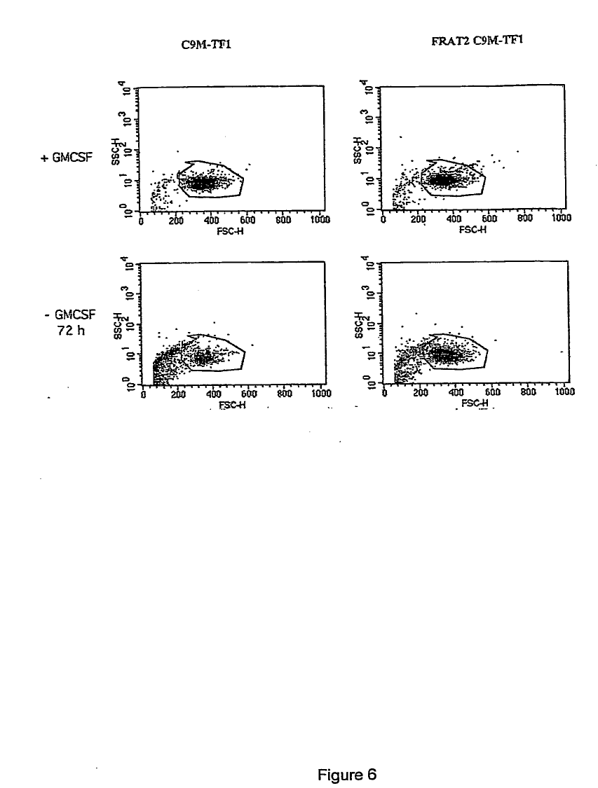

FIG. 6 shows that when GMCSF is withdrawn from either FRAT2 C9M-TF1 or C9M-TF1, the cells undergo apoptosis. However the extent of cells undergoing apoptosis is significantly less in the FRAT2 C9M-TF1 relative to the C9M-TF1 cells (28% c.f. 61%, respectively), thus the presence of FRAT2 makes cells refractive to apoptosis.

Measurement of effect of GMCSF withdrawal using Alamar Blue

This example demonstrates that the protective effective of FRAT2 expression can also be examined using the addition of Alamar Blue to cultures of cells with and without GMCSF. The addition of Alamar Blue to a culture of cells enables the practitioner to measure the viability of the cells. In brief, the internal environment of the proliferating cell is more reduced than that of non-proliferating cells. Specifically, the ratios of NADPH/NADP, FADH/FAD, FMNH/FMN, and NADH/NAD, increase during proliferation. Compounds such as resazurin, the active component of Alamar Blue, can be reduced by these metabolic intermediates and are useful in monitoring cell proliferation because their reduction is accompanied by a measurable shift in colour. The oxidation-reduction potential of resazurin is +380 mV at pH 7.0, 25° C. Resazurin therefore can be reduced by NADPH (Eo=−320 mV), FADH (Eo−−220 mV), FMNH (Eo=−210 mV), NADH (Eo=−320 mV), as well as the cytochromes (Eo=290 mV to +80 mV). As resazurin accepts electrons from these compounds, it changes from the oxidized indigo blue, non-fluorescing state to the reduced fluorescent pink state resorufin. The reduction of resazurin appears to require uptake into the cell. This observation demonstrates that resazurin is not reduced by compounds in the cell culture medium and implicates metabolism within the cells under study as the site of resazurin reduction. Whether the cellular location of reduction of resazurin is cytoplasmic or mitochondrial has yet to be definitively determined. However, resazurin continues to have great utility in measurements of cell viability and proliferation.

Cultures of both FRAT2 C9M-TF1 or C9M-TF1 cells are washed twice in PBS to remove any residual culture medium, prior to being counted and resuspended in culture medium with GMCSF (2 ng/ml) being present or absent at a concentration of 2×10⁵ cells/ml. One hundred microlitres of cells are pipetted into wells of a 96 well plate such that there are 2×10⁴ cells per well. Cells are cultured at 37° C. in an environment of 5% CO₂.

After 72 h, a 10 μL volume of Alamar Blue is added to all wells and the plate is incubated for 34 hours at 37° C. and 5% CO₂ before finally reading the plate with a BioTek SYNERGY plate reader (Excitation 530125 nm—Emission 590/35 nm). Percentage survival is calculated by comparing the fluorescence of cells cultures in the absence of GMCSF to their cognate cultures cultured in the presence of GMCSF.

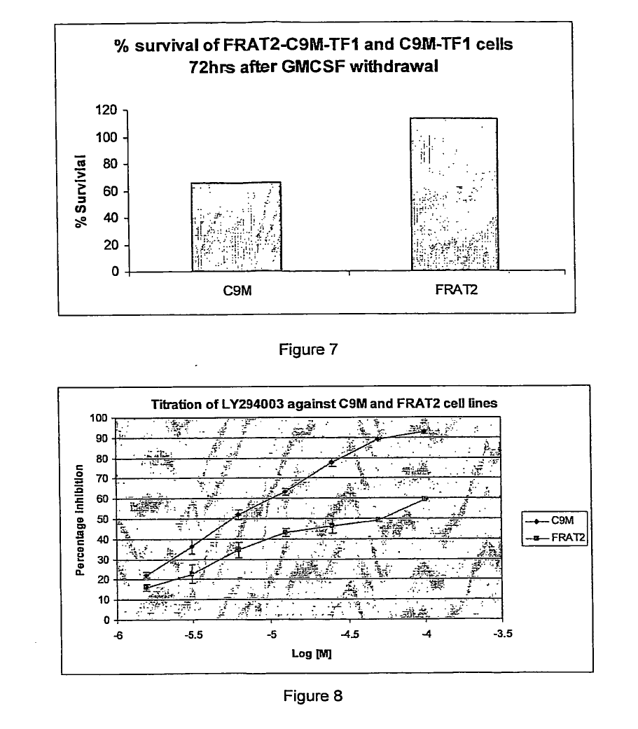

FIG. 7 shows that removal of GMCSF from culture medium in C9M-TF1 cells induces apoptosis, such that the percentage of cells surviving is only approximately 60% of their cognate cells in the presence of GMCSF. In contrast, FRAT2 C9M-TF1 appear to be resistant to apoptosis induced by GMCSF withdrawal as after 72 h, there is not a reduction in the percentage of viable cells, as determined by Alamar Blue conversion.

### Measurement of Effect of the PI3 Kinase Inhibitor LY294002 Using Alamar Blue

This example describes an assay to examine the effect of the PI3K inhibitor LY294002 on cell survival for both FRAT2-C9M-TF1 or C9M-TF1 cells, as determined by Alamar Blue. The growth factor GMCSF mediates its survival effect through the PI3/AKT pathway. Consequently, inhibitors of this pathway will increase apoptosis in pathway-dependent cell lines.

Both FRAT2-C9M-TF1 or C9M-TF1 are split 24 hours prior to drug treatment in order to bring the cultures into the log phase of growth. On the day of the assay the cells are plated directly into a 96 well microtitre plates containing 10 uL volumes of a serial dilutions of LY294002, such that there was a final cell number of 5000 cells per well in 90 uL volumes thus reducing the concentration of the compounds to their final test concentration. The plates are incubated for 72 h at 37° C. and 5% CO₂ after which time they are assayed by Alamar Blue.

FIG. 8 shows the results obtained from one such Control Plate where the PI3-Kinase inhibitor LY294002 was titrated against C9M-TF1 cells and FRAT2-C9M-TF1 cells illustrating that FRAT2 expressing cells are very much more resistant to the effects of LY294002 than are their C9M counterparts. The EC50 for FRAT2 cells is approximately 50 uM whereas the value for C9M cells is approximately 6 uM. The overexpression of FRAT2 leads to the stabilization β-catenin through disassociation of GSK3β from Axin and through the inhibition of β-catenin phosphorylation which results in an increased level of β-catenin in the nucleus of the cell where it forms a complex with the HMB box transcription factors LEF and TCF as well as with CREB Binding Protein. This association of β-catenin with the LEF/TCF transcription factors promotes the expression of a large number of genes, many of which have been identified and shown to be important in the development and progression of colorectal carcinoma. As LY294002 inhibits the phosphorylation of AKT/PKB by PI3-Kinase, AKT/PKB is no longer able to inhibit GSK3β and thus prevent β-catenin phosphorylation, leading to the degradation of β-catenin and the subsequent induction of apoptosis. The increased survival of the FRAT2 overexpressing cell line indicates that the cells are using a particular pathway that is very commonly associated with colon cancer. Consequently, identifying hit molecules that induce apoptosis in the FRAT2 cells suggests that the molecules may be targeting that pathway.

### Example 5

### Primary High—Throughput Screen of the BioFocus SoftFocus SFK Directed Libraries

The current example describes how the candidate compounds were screened against a number of cell lines, including both FRAT2-C9M-TF1 and C9M-TF1 cells.

Primary High-Throughput Screen of Compounds in Adherent cells

Adherent cells are plated at a pre-determined seeding density (Table 1) in flat-bottom 96 well plates 24 hours prior to drug treatment. The cell lines are plated in 100 μl volumes in each well. Cells are plated in all wells apart from those in Column 12 which contain 100 μL normal culture medium alone. These plates will be known as the Test Plates. The Test Plates are labelled with a Daughter Plate ID.

Daughter Plates containing 10 μl of a 10 μM solution of test compound selected from among the candidate compounds stored at ˜80° C. are thawed at room temperature. A 10 μL volume of 0.1% DMSO in PBS is added to all wells of Column 12 and the wells in Row A-F of Column 1. A 10 μl volume of a pre-determined concentration of the P13-Kinase inhibitor LY294002 is added to wells in Row G and H of Column 1. A 90 μl volume of the appropriate cell culture medium is then added to each well and mixed by repeated aspiration/dispensing. The compounds are at a final test concentration of 10 μM.

Once the Daughter Plates have been prepared the media is removed from all wells of the cell culture plate using an 8-Channel Vacuum Aspirator. The contents of the Daughter Plates are then transferred to the appropriate wells of their relevant Test Plates and the Test Plates are incubated for 72 h at 37° C. and 5% CO₂. At the end of this period Test Plates are assayed by Alamar Blue. A 10 μl volume of Alamar Blue is added to all wells of the Test Plate. The Test Plates are incubated for 34 h at 37° C. and 5% CO₂ and finally read using the BioTek SYNERGY plate reader (Excitation 530125 nm-Emission 590135 nm). Percentage inhibition is determined using Microsoft Excel spreadsheet calculations.

### Primary High-Throughput Screen of Compounds in Suspension Cells

Suspension cells are split 24 hours prior to drug treatment in order to bring the cultures into the log phase of growth. On the day of the assay the cells are plated directly into the Daughter Plates at the appropriate pre-determined seeding density (Table 1) in 90 μl volumes, the concentration of the test compounds thus being reduced to 10 μM. Cells are plated in all wells apart from those in Column 12 which contain 901 normal culture medium alone. The plates are incubated for 72 h at 37° C. and 5% CO₂ after which time they are assayed by Alamar Blue as per the preceding example.

TABLE 1

| Cell Line | Adherent/Suspension | Seeding density (cells per well) |
|---|---|---|
| C9M | Suspension | 5,000 |
| FRAT2 | Suspension | 5,000 |
| PCS | Adherent | 2,500 |
| A549 | Adherent | 5,000 |
| U1810 | Adherent | 5,000 |
| NCI/ADR-RES | Adherent | 5,000 |
| MCF7 | Adherent | 1,000 |
| MCF10A | Adherent | 1,000 |
| HT29 | Adherent | 1,000 |
| SW480 | Adherent | 10,000 |
| CACO2 | Adherent | 10,000 |
| LS123 | Adherent | 10,000 |

The results of the primary high-throughput screen of the compounds are listed in Table 2. The figures appearing in the columns are the percentage kill achieved by that compound at 10 μM. Negative values indicate that the compounds induced a positive effect on growth characteristics.

TABLE 2

| | CELL LINE | | | | | | | | | | | | |
|---|---|---|---|---|---|---|---|---|---|---|---|---|---|
| | | | | | | | | | | | | NCI | |
| | | | | | | | | | | | | ADR | |
| COMPOUND | C9M | C9M | FRAT2+ | FRAT2− | HT29 | LS123 | SW480 | CAC02 | A549 | U1810 | MCF7 | RES | PC3 |
| 1 | −1 | 2 | 28 | 54 | 58 | 91 | 33 | 61 | 34 | 15 | 68 | 58 | −4 |
| 2 | −2 | 0 | 33 | 57 | 60 | 91 | 68 | 66 | 18 | 23 | 68 | 73 | 4 |
| 3 | 8 | −12 | 40 | 76 | 80 | 95 | 64 | 58 | 13 | 15 | 85 | 66 | −20 |
| 4 | 1 | 1 | 36 | 71 | 71 | 92 | 53 | 35 | 12 | 16 | 64 | 67 | −4 |
| 5 | 1 | −11 | 42 | 61 | 75 | 87 | 62 | 78 | 10 | 24 | 87 | 63 | −92 |
| 6 | −1 | −11 | 33 | 59 | 20 | 72 | 3 | 37 | 6 | 11 | 64 | 51 | −94 |
| 7 | 9 | 23 | 27 | 53 | 40 | 82 | 44 | 32 | 5 | 18 | 68 | 63 | −16 |
| 8 | 7 | −4 | 39 | 68 | 78 | 92 | 71 | 71 | −3 | 68 | 83 | 85 | 56 |
| 9 | 9 | −5 | 15 | 62 | 69 | 80 | 58 | 65 | 7 | 52 | 76 | 64 | 9 |
| 10 | 14 | −8 | 16 | 71 | 68 | 89 | 51 | 25 | 8 | 16 | 77 | 52 | −45 |
| 11 | 1 | −6 | 35 | 71 | 25 | 22 | −6 | 61 | 5 | 13 | 22 | 18 | −85 |
| 12 | 0 | 0 | 19 | 52 | −5 | 31 | −22 | 17 | 4 | 18 | 36 | −12 | −45 |
| 13 | −1 | −1 | 38 | 61 | −13 | 55 | −4 | 27 | 18 | 13 | 56 | 28 | −37 |
| 14 | 45 | 27 | 38 | 50 | 51 | 13 | 10 | 46 | 26 | 2 | 26 | 2 | −4 |

Abbreviations:
C9M: Derived from TF1 (Human erythroleukaemia)
FRAT2+ FRAT2 overexpresing TF1 (plus GMCSF)
FRAT2− FRAT2 overexpresing TF1 (minus GMCSF)
HT29 Colorectal adenocarcinoma
LS123 Colon adenocarcinoma (Dukes' type B)
SW480 Colon adenocarcinoma (Dukes' type B)
CACO2 Colon adenocarcinoma
A549 Non Small Cell Lung Carcinoma
U1810 Non Small Cell Lung Carcinoma
MCF7 Mammary gland adenocarcinoma
NCI ADR RES Breast adenocarcinoma (pleural effusion)
PCS Prostate carcinoma (bone metastasis)

### Example 6

### Determination of the EC₅₀ for the Compounds Across Cancer and Normal Cells

This Example describes how the EC₅₀ was calculated for the claimed compounds across a breadth of cell lines including the C9M-TF-1 and FRAT2-C9M-TF1, and the colon cancer cell line HT29 and the breast cancer cell line MCF7. Also included in this example is the EC₅₀ of the compounds across the non-transformed breast cell line MCF10A, which have a significantly higher EC₅₀ than their transformed counterparts. This demonstrates that the claimed compounds are more active in cancer cells than their non cancerous counterparts.

### Titration of Selected Compounds

On the day of the assay compounds are used to prepare dilution series on Titration Plates which are plated at ten times their final screening concentration in 10 μl aliquots.

For adherent cell lines plated 24 hours previously, a 90 μl volume of the appropriate cell culture medium is then added to each well of the Titration Plates and mixed by repeated aspiration/dispensing, thus taking the compounds to their final test concentration. Once the Titration Plates have been prepared the media is removed from all wells of the cell culture plate and the contents of the Titration Plates are then transferred to the appropriate wells of their relevant Test Plates and incubated for 72 h at 37° C. and 5% CO₂ after which time they are assayed by Alamar Blue. Using a combination of Microsoft Excel and Graph Pad Prism, EC50s are determined.

### Titration of Active Compounds in Suspension Cells

Suspension cells are split 24 hours prior to drug treatment in order to bring the cultures into the log phase of growth. On the day of the assay the cells are plated directly into the Titration Plates at the appropriate pre-determined seeding density (Table 1) in 90 μl volumes thus reducing the concentration of the compounds to their final test concentration. The plates are incubated for 72 hours at 37° C. and 5% CO₂ after which time they are assayed by Alamar Blue.

Determination of the EC₅₀ values

The EC₅₀ values are calculated using the values obtained from the Alamar Blue readout which are converted into a “Percentage of the Untreated Control” and using Graph Pad Prism these values are plotted against the logarithm of the test concentration. Using a non-linear regression analysis a sigmoidal dose-response curve is generated from which the EC₅₀ is determined.

Table 3 indicates the EC₅₀ values for example compounds when titrated against a range of cell lines. As can be seen in Table 3, the EC50 of C9M-TF1 cells for the indicated compounds was significantly greater (range 2-9 fold greater) than the EC₅₀ values for the engineered cells expressing FRAT2, screened either in the presence (FRAT2+) or absence (FRAT−) of growth factor, confirming our earlier observation from the primary screen that FRAT2-C9M-TF1 are more sensitive to the indicated compounds. For comparison, the EC₅₀ of both cells lines to the phosphoinositide 3-kinase (PI3K) inhibitor LY294002 is also given. However in this experiment the FRAT2-C9M-TF1 cells are more resistant to LY294002, as demonstrated by the significantly different EC₅₀'s concentrations (approx. 8.5 fold difference).

Also described in the table are the EC₅₀ concentrations for the indicated compounds for a transformed colon cell line HT29 and a transformed breast cell line MCF7. The EC₅₀ for the breadth of compounds range from 0.7 μM to 4 μM in the cell based assay.

Lastly, the table lists the EC50 for the compounds on the non transformed breast cell line MCF10A. As can be seen, the EC₅₀ for the compounds are approximately 6-10 fold greater in MCF10A cells than for their transformed counterparts MCF7, thus these compounds are more active in transformed cancer cells than normal cells.

TABLE 3
EC₅₀ values for example compounds when titrated against a selection of cell lines.

| | EC₅₀ (μM) | | | | | | | | | | |
|---|---|---|---|---|---|---|---|---|---|---|---|
| | Com- | Com- | Com- | Com- | Com- | Com- | Com- | Com- | Com- | Com- | |
| | pound | pound | pound | pound | pound | pound | pound | pound | pound | pound | |
| CELL LINE | 1 | 2 | 3 | 6 | 7 | 9 | 4 | 5 | 8 | 10 | LY294002 |
| C9M | 26.00 | 24.00 | 5.62 | 10.00 | 20.42 | 17.78 | 21.38 | 16.60 | 17.78 | 22.91 | 6.03 |
| FRAT2+ | 12.88 | 7.08 | 2.24 | 5.13 | 5.01 | 2.04 | 5.62 | 5.75 | 5.75 | 4.68 | 51.29 |
| FRAT2− | 11.75 | 6.31 | 4.17 | 5.75 | 6.61 | 3.39 | 2.75 | 3.63 | 5.62 | 5.37 | 75.86 |
| HT29 | 3.98 | 4.37 | 0.74 | 1.41 | 4.07 | 2.00 | 1.58 | 1.58 | 1.51 | 1.78 | 5.37 |
| MCF7 | 3.80 | 2.24 | 0.83 | 1.32 | 2.34 | 1.70 | 1.78 | 1.51 | 1.78 | 1.86 | 1.07 |
| MCF10A | 18.62 | 20.42 | 7.24 | 23.44 | 11.48 | 12.88 | 14.45 | 12.88 | 6.17 | 20.42 | 12.88 |
| HCT116 | | 3 | 2.2 | | | | | | | | 21 |
| LoVo | | 2.2 | 2.2 | | | | | | | | 3 |

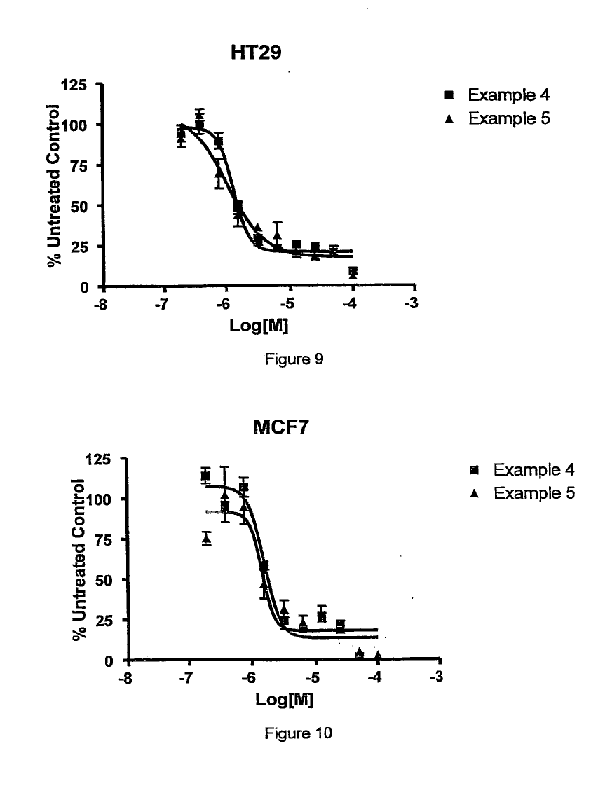

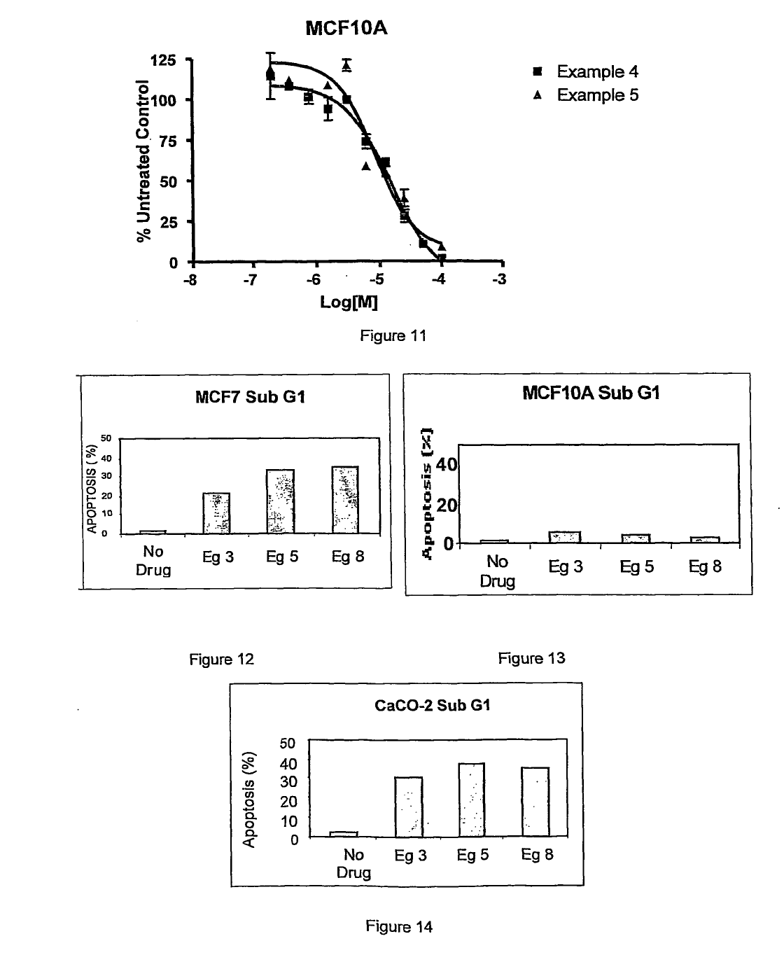

FIGS. 9, 10 and 11 show dose-response curves generated for compounds of examples 4 and 5. FIGS. 19 and 20 show dose-response curves generated for compounds of examples 2 and 3.

### Example 7

### Claimed Compounds Induce Cell Death through Apoptosis

This Example demonstrates that the indicated molecules are able to induce cell death through apoptosis. In the first assay sub G1 analysis is used, the principal of which is based on the fact that as cells undergo apoptosis, their DNA is fragmented and lost from the cell. Thus, measurement of the amount of DNA in a cell will identify cells that have less DNA and are undergoing apoptosis. The second way we demonstrate that the mechanism of cell death is via apoptosis is by examining the percentage of cells that have a compromised mitochondrial polarization potential. Measurements of whole-cell potentiometric dye fluorescence have indicated that mitochondrial polarization is reduced before the appearance of apoptotic nuclear changes in a variety of blood, hepatic, and immune cell models (Susin et al, 1996; Apoptosis 1:231-242). Consequently, by measuring the mitochondrial polarization of cells treated with and without the molecules, one can examine the percentage of cells that are undergoing apoptosis.

### 1. Sub G1 Analysis of Apoptosis

Examination of the DNA content of a cell can determine the percentage of cells that are undergoing apoptosis, since DNA fragmentation is a hallmark of apoptosis. Cells in G1 have a diploid amount of DNA or ‘2n’, those with a DNA quantity>2n are in the synthesis or ‘S’ phase of the cell cycle, those with ‘4n’ are in the G2M phase of the cell cycle and are about to divide to give 2 cells. However cells undergoing apoptosis have<2n or a Sub G1 amount of DNA since during the process of apoptosis, the DNA fragments and leaks out of the nucleus of the cell; therefore by quantifying the percentage of cells with a sub G1 amount of DNA, one can quantify the percentage of apoptotc cell is the population. Quantification of the percentage of cells in each phase of the cell cycle is achieved by permeabilising the cell suspension and staining the DNA in the nucleus of each cell with Propidium Iodide (PI).

Propidium Iodide is a fluorescent stain and therefore the intensity of the fluorescence detected by the flow cytometer correlates with the amount of DNA within that cell. By analysing cells exposed to the indicated molecules, the percentage of the cells with a Sub G1 profile and hence the percentage of cells undergoing apoptosis can be ascertained.

The indicated cell lines are plated in a 6 well plate such there is 100,000 cells per well in full culture medium. Cells are treated with the indicated small molecule at a final concentration of 25 μM for 72 h. After this time the cells are stained by resuspending the cells in staining buffer (0.1% Sodium Citrate, 0.1% TritonX-100, 200 μl of Propidium Iodide at 5 mg/ml made up to 20 mls in PBS) for 24 hours at 4° C. in the dark. The PI stained cells are then acquired by the flow cytometer. Analysis of FL2 fluorescence is performed on Cell Quest software to allow quantification of the Sub-G1 phase of the cell.

### 2. JC-1 Analysis of Mitochondrial Membrane Depolarisation

Analysis of the Mitochondrial membrane potential of a cell population determines the percentage of Apoptotic and viable cells within a population. Mitochondrial Membrane Potential is quantified utilising the JC-1 Dye acquired from Molecular Probes and used according to manufacturers instructions.

The indicated cell lines are plated in a 6 well plate such there is 100,000 cells per well in full culture medium. Cells are exposed for the indicated small molecule at a final concentration of 25 μM for 72 h.

Cells are harvested and incubated with the JC1 dye as per manufacturers protocol. When this dye is incubated with viable cells its remains in an aggregate form that fluoresces red on activation with an argon laser. However when apoptosis is occurring the JC-1 dye shifts to a predominantly monomer form that fluoresces green on activation. Therefore a shift from a red to a red/green or green fluorescent population indicates an apoptotic population. Analysis of fluorescence is performed on Cell Quest software to allow quantification of the apoptosis occurring in the population of cells examined. The percentage of cells undergoing apoptosis post treatment with molecules is compared directly to apoptosis induced in control wells.

FIGS. 12, 13 and 14 show apoptosis modulation of Cancer Cells by example compounds of the invention as determined by sub G1 analysis.

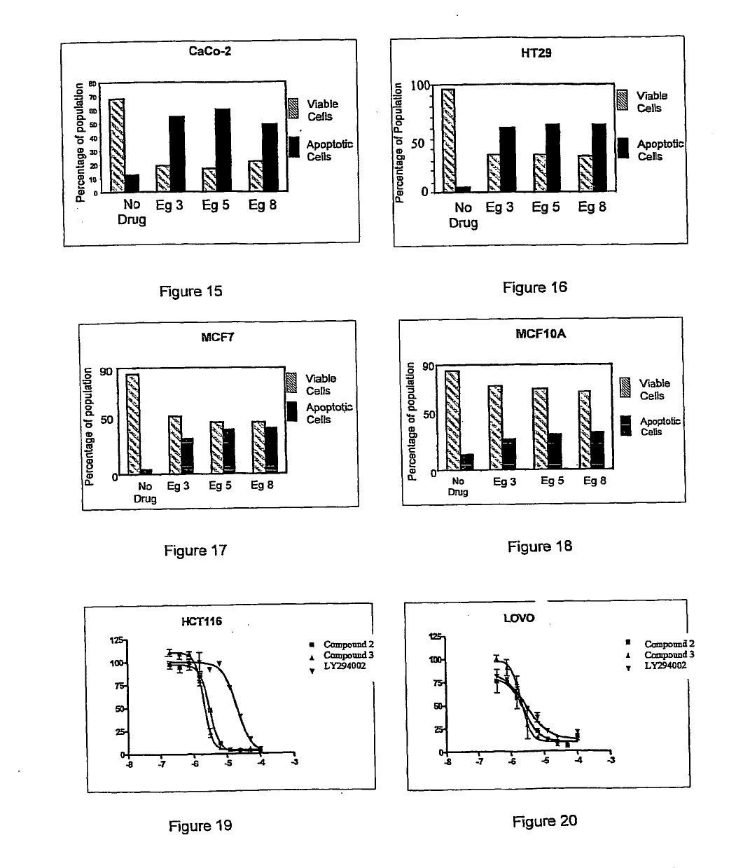

FIGS. 15 to 18 show apoptosis modulation of cancer cells by example compounds as determined by JC-1 analysis

Apoptosis in the cell lines HT29, CaCO2, MCF7 and MCF10 as detected by Sub G1 and JC1 Analysis. All assays were performed following 72 h of treatment with 2511M of the indicated small molecule compound. A) The Sub G1 Analysis measures DNA Fragmentation, a characteristic of cells undergoing apoptosis. The percentage Sub G1 correlates positively with cells undergoing apoptosis. As can be seen in FIG. 12-14, treatment of cells with the compounds increases the percentage of cells undergoing apoptosis as demonstrated by the increase in cells having a Sub G1 profile. Of note however is the fact that MCF10A cells have reduced number of cells with a sub G1 profile, relevant to its transformed counterpart MCF7 (FIG. 13 vs. FIG. 12), demonstrating that the compounds are able to selectively induce apoptosis transformed cells over non transformed cells B) The mitochondrial potential of cells treated with the compounds is also reduced as demonstrated by JC-1 staining. Thus as can be seen in FIG. 15-18, the percentage of the population with their mitochondrial potential in tact(Hatched bar) is greatly reduced following treatment with indicated compound, whilst the percentage of cells with a compromised mitochondrial potential is increased (dark bar), indicating that treatment of cells with compounds induces apoptosis. Again however, one can see that MCF10A cells appear to be more resistant than its transformed counterpart (FIG. 18 vs. FIG. 17).

Therefore, by examining cells treated with the said compounds, and examining the effect of the compound on the process of cell death by either DNA fragmentation (as determined by sub-G1) or mitochondrial integrity (as examined by JC-1 analysis), one can conclude that the compounds kill the cancer cells through the process of apoptosis. All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

1. A compound of the formula (I) 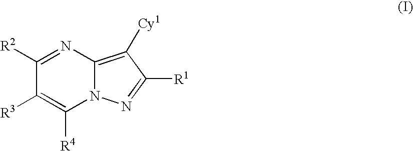
wherein
Cy¹ is an optionally substituted mono or bicyclic aromatic group of from 5 to 10 ring members and 1 to 10 carbon atoms, optionally having from 1 to 5 heteroatoms independently selected from sulphur, oxygen and nitrogen;
R¹, R² and R³ are each independently selected from hydrogen, hydroxyl, halogen, alkyl of 1 to 6 carbon atoms, haloalkyl of 1 to 6 carbon atoms comprising from 1 to a maximum number of halogen atoms, cycloalkyl of 3 to 8 carbon atoms, alkoxy of 1 to 6 carbon atoms, cycloalkoxy of 3-8 carbon atoms, haloalkoxy of 1 to 6 carbon atoms comprising from 1 to a maximum number of halogen atoms, thioalkyl of 1 to 6 carbon atoms, sulfoxoalkyl of 1 to 6 carbon atoms, sulfonoalkyl of 1 to 6 carbon atoms, aryl of 6 to 10 carbon atoms, —COR⁵, —CO₂R⁵, —NO₂, —CONR⁵R⁶, —NR⁵R⁶ or —N(R⁵)COR⁶, —CN, a 5 or 6-membered heterocyclic ring having from 1 to 4 heteroatoms selected from O, N or S; —NO₂, —NR⁵R⁶, —CHFCN, —CF₂CN, alkynyl of 2 to 7 carbon atoms, or alkenyl of 2 to 7 carbon atoms; wherein the alkyl, heterocyclic ring, alkenyl or alkynyl moieties are optionally substituted with hydroxyl, —CN, halogen, alkyl of 1 to 6 carbon atoms, alkoxy of 1 to 6 carbon atoms, —COR⁵, —CO₂R⁵, —NO₂, —CONR⁵R⁶, —NR⁵R⁵ or —N(R⁵)COR⁶;
R⁴ is a group of the formula —NH—CH₂—Cy²;
R⁵ and R⁵ are each, independently hydrogen, alkyl of 1 to 6 carbon atoms or aryl of 6-10 carbon atoms;
Cy² is an optionally substituted cyclic group;
or a pharmaceutically acceptable salt form thereof;
for use as a medicament.

2. A compound of the formula (II) 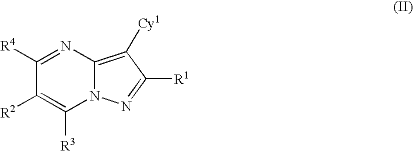
wherein
Cy¹ is an optionally substituted mono or bicyclic aromatic group of from 5 to 10 ring members and 1 to 10 carbon atoms, optionally having from 1 to 5 heteroatoms independently selected from sulphur, oxygen and nitrogen;
R¹, R² and R³ are each independently selected from hydrogen, hydroxyl, halogen, alkyl of 1 to 6 carbon atoms, haloalkyl of 1 to 6 carbon atoms comprising from 1 to a maximum number of halogen atoms, cycloalkyl of 3 to 8 carbon atoms, alkoxy of 1 to 6 carbon atoms, cycloalkoxy of 3-8 carbon atoms, haloalkoxy of 1 to 6 carbon atoms comprising from 1 to a maximum number of halogen atoms, thioalkyl of 1 to 6 carbon atoms, sulfoxoalkyl of 1 to 6 carbon atoms, sulfonoalkyl of 1 to 6 carbon atoms, aryl of 6 to 10 carbon atoms, —COR⁵, —CO₂R⁵, —NO₂, —CONR⁵R⁶, —NR⁵R⁶ or —N(R⁵)COR⁶, —CN, a 5 or 6-membered heterocyclic ring having from 1 to 4 heteroatoms selected from O, N or S; —NO₂, —NR⁵R⁶, —CHFCN, —CF₂CN, alkynyl of 2 to 7 carbon atoms, or alkenyl of 2 to 7 carbon atoms; wherein the alkyl, heterocyclic ring, alkenyl or alkynyl moieties are optionally substituted with hydroxyl, —CN, halogen, alkyl of 1 to 6 carbon atoms, alkoxy of 1 to 6 carbon atoms, —COR⁵, —CO₂R⁵, —NO₂, —CONR⁵R⁶, —NR⁵R⁵ or —N(R⁵)COR⁶;
R⁴ is a group of the formula —NH—Cy²;
R⁵ and R⁶ are each, independently hydrogen, alkyl of 1 to 6 carbon atoms or aryl of 6-10 carbon atoms;
Cy² is an optionally substituted cyclic group;
or a pharmaceutically acceptable salt form thereof;
or use as a medicament.

3. A compound according to claim 1 or 2 wherein Cy¹ is an optionally substituted phenyl, thiophene (preferably 2-thiophene), pyridine (preferably 4-pyridine) or quinoline (preferably 8-quinoline) group or a pharmaceutically acceptable salt form thereof for use as a medicament.

4. A compound according to any preceding claim wherein Cy¹ is a mono- or disubstituted group, wherein the substituents are independently selected from alkoxy of 1 to 6 carbon atoms, alkyl of 1 to 6 carbon atoms, halogen, —NR¹⁰R¹¹ (wherein R¹⁰ and R¹¹ are each, independently hydrogen or alkyl of 1 to 6 carbon atoms), morpholino, haloalkyl of 1 to 6 carbon atoms comprising from 1 to a maximum number of halogen atoms.

5. A compound according to any preceding claim wherein the substituents are independently selected from methoxy, fluoro, chloro, dimethylamino, morpholino and chloromethyl.

6. A compound according to any preceding claim wherein Cy¹ is selected from 3-methoxyphenyl, 3,4-dimethoxyphenyl, 4-methoxyphenyl, 2,4-dimethoxyphenyl, phenyl, 2-fluorophenyl, 3-(dimethylamino)phenyl, 3-quinolin-8-yl, 6-methoxy-pyridin-3-yl, 4-methyl-thiophen-2-yl, 3-chloro-4-fluorophenyl, 2-(chloromethyl)phenyl, morpholinyl-phenyl and 4-pyridyl.

7. A compound according to any preceding claim wherein R¹, R² and R³ are all hydrogen or a pharmaceutically acceptable salt form thereof for use as a medicament.

8. A compound according to any preceding claim wherein Cy² is an optionally substituted mono or bicyclic aromatic group of from 5 to 10 ring members and 1 to 10 carbon atoms, optionally having from 1 to 5 heteroatoms independently selected from sulphur, oxygen and nitrogen or a pharmaceutically acceptable salt form thereof for use as a medicament.

9. A compound according to any preceding claim wherein Cy² is an optionally substituted thiophene (preferably 2-thiophene) or pyridine (preferably 2-pyridine) group or a pharmaceutically acceptable salt form thereof for use as a medicament.

10. A compound according to claim 9 wherein Cy² is pyridin-2-yl:

11. A compound which is one of 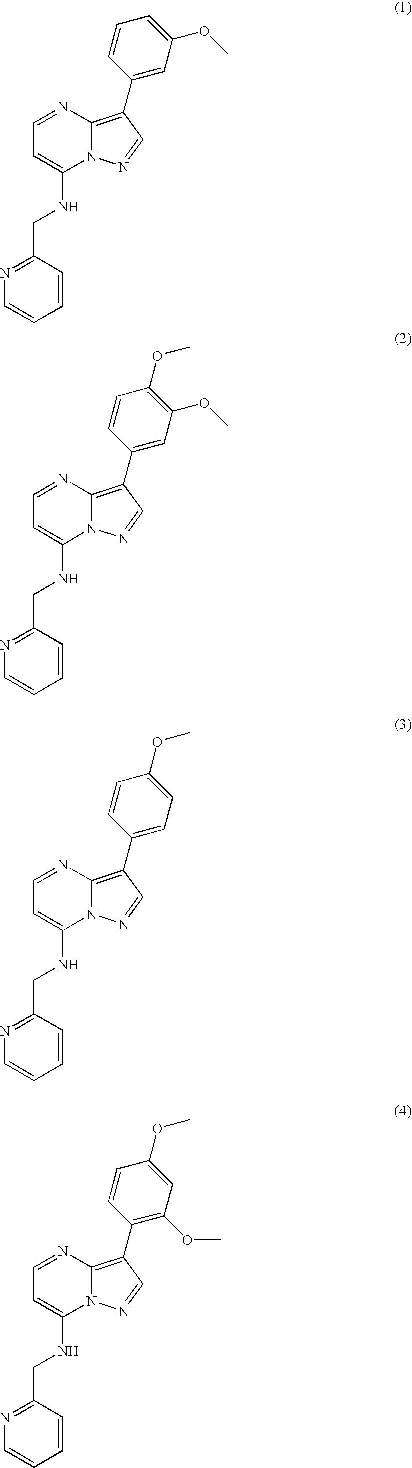 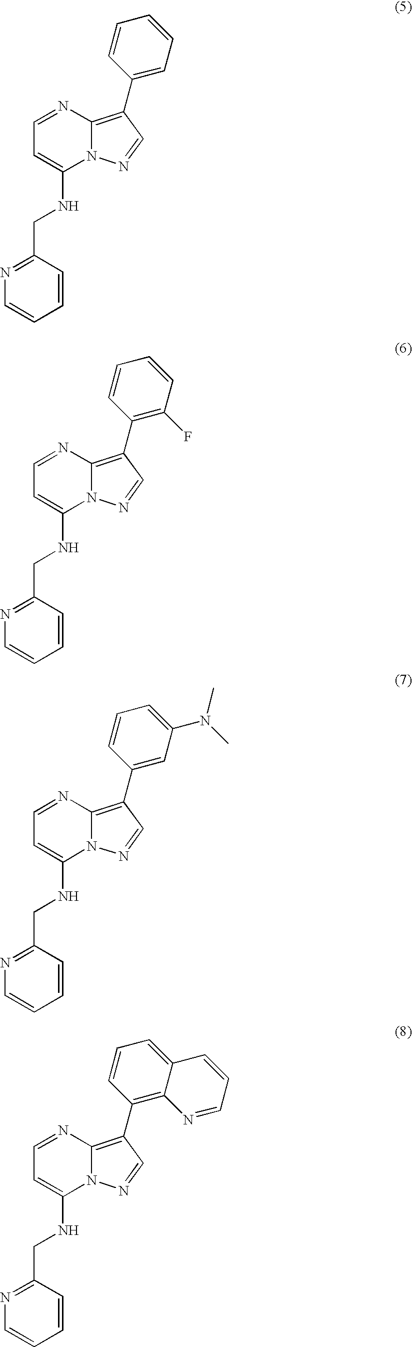 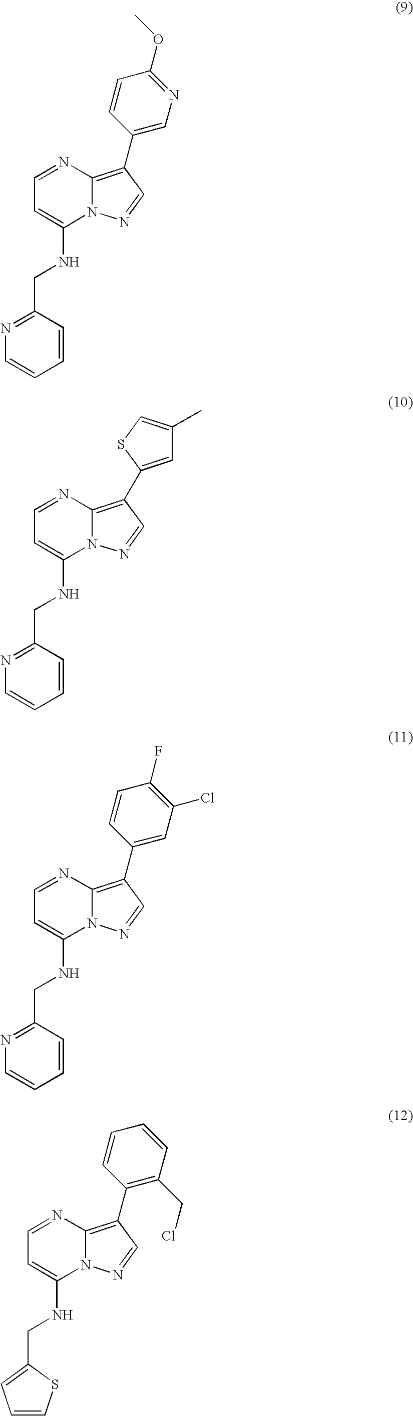 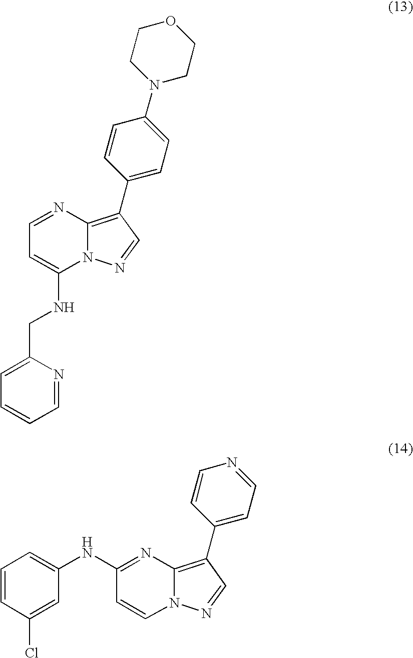
or a pharmaceutically acceptable salt form thereof for use as a medicament.

12. A pharmaceutical composition comprising a compound as claimed in any one of claims 1 to 11 together with at least one pharmaceutically acceptable carrier.

13. A method of treating, inhibiting or preventing cancer in a mammal in need thereof comprising providing to said mammal an effective amount of a compound as claimed in any one of claims 1 to 11.

14. The method of claim 13 wherein the cancer is associated with an anomaly in the Wnt signalling pathway.

15. The method of claim 13 wherein the cancer is cancer of the colon.

16. The method of claim 13 wherein the cancer is cancer of the breast.

17. A method of inducing, inhibiting or modulating apoptosis in a mammal comprising providing to said mammal an effective amount of a compound as claimed in any one of claims 1 to 11.

18. A method of treating, inhibiting or preventing a disease associated with apoptosis in a mammal in need thereof comprising providing to said mammal an effective amount of a compound as claimed in any one of claims 1 to 11.

19. Use of a compound as claimed in any one of claims 1 to 11 in a process for the preparation of a medicament for treating, inhibiting or preventing cancer.

20. Use according to claim 19 wherein the cancer is associated with an anomaly in the Wnt signalling pathway.

21. Use according to claim 19 wherein the cancer is cancer of the colon.

22. Use according to claim 19 wherein the cancer is cancer of the breast.

23. Use of a compound as claimed in any one of claims 1 to 11 in a process for the preparation of a medicament for the induction, inhibition or modulation of apoptosis.

24. Use of a compound as claimed in any one of claims 1 to 11 in a process for the preparation of a medicament for treating, inhibiting or preventing a disease associated with apoptosis.

25. A method for detecting apoptosis-modulating activity in a candidate compound comprising the steps of
i) providing a reference cell line;
ii) providing the reference cell line transformed to overexpress the FRAT2 gene (transformed cell line);
iii) incubating a candidate compound with a) the reference cell line, b) the transformed cell line in the absence of GM-SF, and c) the transformed cell line in the presence of GM-CSF;
iv) quantifying the proportion of cells killed in cases a), b) and c);
v) comparing the proportion of cells killed in cases a), b) and c); wherein the proportion killed in c) being greater than the proportion killed in a) and the proportion killed in b) being greater than the proportion killed in c) being indicative of apoptosis-modulating activity.

26. The method according to claim 25 wherein the reference cell line is Human erythroleukaemia (C9M TF1).


SEQUENCE LISTING (16 sequences)

SEQ ID NO: 1 - DNA, 26 bp, Artificial Sequence
  Primer FRAT2 forward
ccaagcttggacggggggccatgccg

SEQ ID NO: 2 - DNA, 22 bp, Artificial Sequence
  Primer FRAT2 reverse
ggcctgcgtcagagcaaggagc

SEQ ID NO: 3 - DNA, 20 bp, Artificial Sequence
  Primer T7 forward
taatacgactcactataggg

SEQ ID NO: 4 - DNA, 17 bp, Artificial Sequence
  Primer BGH reverse
tagaaggcacgtcgagg

SEQ ID NO: 5 - DNA, 49 bp, Artificial Sequence
  Primer T7-AttB1 forward
ggggacaagtttgtacaaaaaagcaggcttaatacgactcactataggg

SEQ ID NO: 6 - DNA, 47 bp, Artificial Sequence
  Primer BGH-attB2 reverse
ggggaccactttgtacaagaaagctgggttagaaggcacagtcgagg

SEQ ID NO: 7 - DNA, 19 bp, Artificial Sequence
  Primer QFRAT2 forward
agcgccgatggacccaagc

SEQ ID NO: 8 - DNA, 20 bp, Artificial Sequence
  Primer QFRAT2 reverse
agggcaatgcggtcaggtcc

SEQ ID NO: 9 - DNA, 23 bp, Artificial Sequence
  Primer Qbeta-catenin forward
atcccactaatgtccagcgtttg

SEQ ID NO: 10 - DNA, 20 bp, Artificial Sequence
  Primer Qbeta-catenin reverse
tgcaaggtcccagcggtaca

SEQ ID NO: 11 - DNA, 18 bp, Artificial Sequence
  Primer Qc-myc forward
ccgcccctgtcccctagc

SEQ ID NO: 12 - DNA, 21 bp, Artificial Sequence
  Primer Qc-myc reverse
tccgggtcgcagatgaaactc

SEQ ID NO: 13 - DNA, 20 bp, Artificial Sequence
  Primer QcyclinD1 forward
cgcgccctcggtgtcctact

SEQ ID NO: 14 - DNA, 20 bp, Artificial Sequence
  Primer QcyclinD1 reverse
gcggccaggttccacttgag

SEQ ID NO: 15 - DNA, 21 bp, Artificial Sequence
  Primer Qcox2 forward
tgcctgatgattgcccgactc

SEQ ID NO: 16 - DNA, 23 bp, Artificial Sequence
  Primer Qcox2 reverse
tggccctcgcttatgatctgtct